(19) United States
(12) Patent Application Publication
(10) Pub. No.: US 2019/0025285 A1
(43) Pub. Date: Jan. 24, 2019

(54) METHODS OF DETERMINING RELATIVE POTENCY OF GLATIRAMER ACETATE AND USE THEREOF IN PREPARING A BATCH OF GLATIRAMER ACETATE AS ACCEPTABLE FOR PHARMACEUTICAL USE

(71) Applicant: InSight Biopharmaceutical Ltd., Rechovot (IL)

(72) Inventors: Ilana COHEN, Nes Ziona (IL); Dalit HECHT, Rehovot (IL)

(21) Appl. No.: 15/550,794

(22) PCT Filed: Feb. 12, 2015
(86) PCT No.: PCT/IL2015/050164

§ 371 Date: Feb. 28, 2018

(51) Int. Cl. G01N 33/50; A61K 31/785; A61K 9/00

(52) U.S. Cl. CPC G01N 33/505; A61K 31/785; G01N 2650/00; G01N 2333/55; A61K 9/0019

(57) ABSTRACT

A method of determining relative potency of glatiramer acetate (GA) is provided. Accordingly there is provided a method comprising: immunizing a mammal with Poly-YAK copolymer (pYAK); preparing a primary culture of T-cells from the immunized mammal; stimulating samples of the primary culture of T-cells with a reference standard batch of GA or a batch of GA; determining a stimulation parameter of the cells in each sample following a predetermined incubation time; and comparing the stimulation parameter in the cells so as to determine the relative potency of the batch of GA. Also provided are methods of preparing a batch of GA as acceptable for pharmaceutical use and treating multiple sclerosis (MS) in a subject.

### FIELD AND BACKGROUND OF THE INVENTION

The present invention, in some embodiments thereof, relates to methods of determining relative potency of glatiramer acetate and use thereof in preparing a batch of glatiramer acetate as acceptable for pharmaceutical use.

Multiple sclerosis (MS) is an autoimmune inflammatory, demyelinating disease of the central nervous system. Activated T-cells, recognizing self-epitopes derived from myelin sheets, are a key element in disease progression.

Glatiramer acetate (GA, also known as Copolymer-1, Copolymer 1, Cop-1 or COPAXONE®), is an FDA (as well as other regulatory agencies) approved drug for the treatment of MS. Initially, GA was shown to suppress Experimental Autoimmune Encephalomyelitis (EAE, an experimental model for MS) in various animal species. Later on, GA was shown to reduce the frequency of relapses, to reduce the burden and activity of disease on MRI and, in few instances, to slow the progression of disability in relapsing-remitting MS clinical trials (Aharoni, Autoimmunity Reviews (2013) 12: 543-553).

GA is composed of acetate salts of synthetic polypeptides, containing four naturally occurring amino acids, found in myelin basic protein, in a specified average molar fraction of L-glutamic acid: 0.129-0.153; L-alanine: 0.392-0.462; L-tyrosine: 0.086-0.100; and L-lysine: 0.300-0.374. The average molecular weight of GA is 4,700-11,000 daltons [Physician's Desk Reference (PDR)]. Chemically, GA is designated (PDR) L-glutamic acid polymer with L-alanine, L-lysine and L-tyrosine, acetate (salt) and its structural formula is:

(Glu,Ala,Lys,Tyr)ₓ.xCH₃COOH

(C₅H₉NO₄.C₃H₇NO₂.C₆H₁₄N₂O₂.C₉H₁₁NO₃)ₓ.xC₂H₄O₂

### CAS-147245-92-9

The mechanism of action of GA is still not fully elucidated, however current accumulated data from both pre-clinical and clinical data has shown that the biological activity of GA in MS is mediated, at least in part, by immune-modulation of T cell activity; promoting a shift from Tₕ1 pro-inflammatory response to a Tₕ2 anti-inflammatory response. GA related copolymers comprising D amino acids instead of L amino acids, copolymers in which specific amino acids were replaced, or copolymers comprising 3 of the 4 amino acids of GA exhibited different results in their ability to suppress EAE in various animal models [Teitelbaum D. et al. Eur. J. Immunol. (1973) 3: 273-279; Webb C. et al. Immunohistochemistry (1976) 13: 333-337], indicating the specificity of the antigenic recognition is important for GA suppressive activity in MS.

The batch to batch variation necessitates the development of specific, reproducible and robust biological assays (also called bioassays, or potency assays) for qualifying GA activity.

U.S. Pat. Nos. 7,923,215, 7,429,374 and 8,389,228 to Teva disclose methods for measuring the relative potency of a test batch of GA.

Additional related art: Fridkis-Hareli et al. [International Immunology (1999) 11: 635-641].

### SUMMARY OF THE INVENTION

According to an aspect of some embodiments of the present invention there is provided a method of determining relative potency of a batch of glatiramer acetate (GA), the method comprising:
(a) immunizing a mammal with an immunization effective amount of a Poly-YAK copolymer (pYAK) so as to obtain an immunized mammal;
(b) preparing a primary culture of T-cells from the immunized mammal;
(c) individually stimulating samples comprising substantially identical number of cells of the primary culture of T-cells with a predetermined amount of:
   (i) a reference standard (RS) batch of GA; or
   (ii) the batch of GA,
   wherein the predetermined amount of (i) is substantially identical to the predetermined amount of (ii);
(d) determining a stimulation parameter of the cells in each of the samples following a predetermined incubation time; and
(e) comparing the stimulation parameter in the cells of step (c)(i) with the stimulation parameter of the cells of step (c)(ii), so as to determine the relative potency of the batch of GA.

According to some embodiments of the invention the method further comprising generating stimulation curves of the samples incubated with predetermined amount of the RS batch of GA and of the samples incubated with predetermined amount of the batch of GA, so as to determine parallelism.

According to some embodiments of the invention the mammal is a rodent.

According to some embodiments of the invention the rodent is a mouse.

According to some embodiments of the invention the mouse is a female (SJLXBALB/C) F1 mouse.

According to some embodiments of the invention the mouse is 8 to 12 weeks old.

According to some embodiments of the invention the immunization effective amount of pYAK comprises 100 to 250 μg per mammal per boost.

According to some embodiments of the invention the immunizing comprises administering an effective amount of an adjuvant simultaneously with the Poly-YAK copolymer (pYAK).

According to some embodiments of the invention the adjuvant is complete Freund's adjuvant (CFA).

According to some embodiments of the invention the preparing a primary culture of T-cells is effected 3 to 14 days following immunizing the mammal.

According to some embodiments of the invention the T-cells are lymph node cells.

According to some embodiments of the invention the T-cells are spleen cells.

According to some embodiments of the invention the samples of each of the (c)(i) and (c)(ii) comprise at least 2 repeats.

According to some embodiments of the invention the samples of each of the (c)(i) and (c)(ii) comprise at least 3 repeats.

According to some embodiments of the invention the predetermined amount of the RS batch of GA and the batch of GA comprise 2 μg/ml to 80 μg/ml.

According to some embodiments of the invention the stimulation parameter is cytokine secretion.

According to some embodiments of the invention the cytokine is an interleukin.

According to some embodiments of the invention the interleukin is interleukin-2.

According to some embodiments of the invention the predetermined incubation time comprises 17 to 27 hours.

According to an aspect of some embodiments of the present invention there is provided a method of preparing a batch of glatiramer acetate as acceptable for pharmaceutical use, the method comprising:

(a) preparing a batch of GA;
(b) measuring the relative potency of the batch according to the method of the invention; and
(c) qualifying the batch as acceptable for pharmaceutical use if the relative potency so measured in step (b) is between 80% and 125% of the RS batch of GA.

According to some embodiments of the invention the method further comprising generating stimulation curves of the samples incubated with predetermined amount of the RS batch of GA and of the samples incubated with predetermined amount of the batch of GA, and qualifying the batch as acceptable for pharmaceutical use if the stimulation curves are parallel.

According to an aspect of some embodiments of the present invention there is provided a method of preparing a pharmaceutical composition comprising GA, the method comprising:

(a) preparing a batch of GA;
(b) measuring the relative potency of the batch according to the method of the invention; and
(c) preparing the pharmaceutical composition with the batch of GA if the relative potency so measured in step (b) is between 80% and 125% of the RS batch of GA.

According to some embodiments of the invention the method further comprising generating stimulation curves of the samples incubated with predetermined amount of the RS batch of GA and of the samples incubated with predetermined amount of the batch of GA, and preparing the pharmaceutical if the stimulation curves are parallel.

According to an aspect of some embodiments of the present invention there is provided a method of treating multiple sclerosis (MS) in a subject in need thereof, the method comprising injecting into the subject a pharmaceutical composition which comprises GA generated according to the method of the invention, thereby treating MS in a subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flow chart depicting the main steps of the ex-vivo potency assay.

FIG. 2 is a representative log/log graph of IL-2 levels secreted from LNCs extracted from mice immunized with Poly-YAK (YB106) in response to in-vitro stimulation with increasing concentrations of RS demonstrating a dose-dependent response.

FIG. 3 is a log/log graph of IL-2 secretion from LNCs extracted from immunized mice in response to in-vitro stimulation with increasing concentrations of GA reference standard (RS). Mice were immunized with Poly-EAK (YB105, inverted triangles), Poly-YAK (YB106, triangles), Poly-YEA (YB107, diamonds) or Poly-YEK (YB108, squares). Data is presented as average±SD.

FIGS. 4A-F are log/log graphs of IL-2 levels secreted from LNCs extracted from mice immunized with Poly-YAK (YB106) in response to in-vitro stimulation with increasing concentrations of Poly-EAK (YB105, FIG. 4A), Poly-YEA (YB107, FIG. 4B), Poly-YAK (YB106, FIG. 4C), Poly-YEK (YB108, FIG. 4D), short Poly-YEAK (YB109, FIG. 4E) and long Poly-YEAK (YB110, FIG. 4F) in comparison to RS, demonstrating distinct response pattern to each copolymer tested.

FIG. 5A is a log/log graph of IL-2 levels secreted from LNCs extracted from mice 8 (circles), 9 (squares), or 10 (triangles) days following immunization with Poly-YAK (YB106) in response to in-vitro stimulation with increasing concentrations of RS (3.7-80 μg/ml).

FIG. 5B is a log/log graph of IL-2 levels secreted from LNCs extracted from mice 3 (circles), 9 (triangles), or 14 (squares) days following immunization with Poly-YAK (YB106) in response to in-vitro stimulation with increasing concentrations of RS (3.7-80 μg/ml).

FIG. 6A is a log/log graph of IL-2 secretion from LNCs extracted from mice immunized with two different doses of Poly-YAK (YB106), 100 μg/mouse (circles) and 250 μg/mouse (squares), in response to in-vitro stimulation with increasing concentrations of RS.

FIG. 6B is a graph depicting the observed vs. expected relative potency of RS accuracy samples, demonstrating overall assay linearity over the concentration range 50%-200% with R²=0.99.

FIGS. 7A-F are log/log graphs of IL-2 levels secreted from LNCs extracted from mice immunized with Poly-YAK (YB106) in response to in-vitro stimulation with increasing concentrations of COPAXONE® batches P53714 (FIG. 7A), P53791 (FIG. 7B), P53459 (FIG. 7C), P53551 (FIG. 7D), P53889 (FIG. 7E) and P53804 (FIG. 7F) in comparison to RS (COPAXONE®-Teva, batch P53668) demonstrating a similar response pattern for all batches tested.

FIGS. 8A-F are log/log graph of IL-2 levels secreted from LNCs extracted from mice immunized with Poly-YAK (YB106) in response to in-vitro stimulation with increasing concentrations of GA non-commercial batches YB124 (FIG. 8A), YB126 (FIG. 8B), ENG1 (FIG. 8C), ENG2 (FIG. 8D), ENG3 (FIG. 8E) and ENG4 (FIG. 8F), in comparison to RS (COPAXONE®-Teva, batch P53668) demonstrating a similar response pattern for all batches tested.

FIGS. 9A-B are log/log graphs of IL-2 secretion from lymph node cells (LNCs) extracted from GA immunized mice in response to in-vitro stimulation with increasing concentrations of Poly-EAK (YB105, FIG. 9A, triangles), Poly-YAK (YB106, FIG. 9A, diamonds), Poly-YEA (YB107, FIG. 9B, triangles) or Poly-YEK (YB108, FIG. 9B, diamonds) in comparison to GA reference standard (RS, squares).

### DESCRIPTION OF SPECIFIC EMBODIMENTS OF THE INVENTION

The present invention, in some embodiments thereof, relates to methods of determining relative potency of glatiramer acetate and use thereof in preparing a batch of glatiramer acetate as acceptable for pharmaceutical use.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The batch to batch variation of glatiramer acetate (GA), an FDA approved drug for the treatment of MS, necessitates the development of specific, reproducible and robust biological assay (also called bioassays, or potency assay) for qualifying GA activity.

U.S. Pat. Nos. 7,923,215, 7,429,374 and 8,389,228 to Teva teach an ex-vivo potency assay for GA. These patents show that the assay is highly specific to GA in-vitro stimulation and that GA-specific T-cells do not respond to in-vitro stimulation using 3 amino acids copolymers lacking Lysine, Alanine or Tyrosine (Poly-YEA, Poly-YEK and Poly-EAK, respectively).

Whilst reducing the present invention to practice, the present inventors have developed an ex-vivo potency assay. The assay is based on the surprising realization that Poly-YAK, a copolymer containing tyrosine (Y), alanine (A), and lysine (K), which are only three of the amino acids composing GA can mimic the immunogenic activity of GA and as such can be implemented in an ex-vivo potency assay for qualifying GA activity.

The assay for evaluating the biological activity of GA is based on immunization of mice using Poly-YAK followed by in-vitro stimulation with increasing doses of GA and evaluation of T-lymphocyte activation such as by assessing the level of cytokine secretion. Most importantly, the present inventors have uncovered that the Poly-YAK mimic of GA is superior to other 3 amino acids copolymers (Poly-EAK, Poly-YEA and Poly-YEK). This presently developed potency assay comprises a nice slope of the biological response curve that can be used as an accurate and reproducible method for GA biological qualification.

The current invention is surprising in view of previous works on the subject. Briefly, U.S. Pat. Nos. 7,923,215, 7,429,374 and 8,389,228 to Teva show that GA-specific T-cells did not respond to in-vitro stimulation using the aforementioned three amino acid copolymers (see tables 10, 11 and 10, respectively), thus establishing that copolymers comprising 3 of the 4 amino acids of GA cannot mimic GA therefore are not cross reactive with GA.

The work of Fridkis-Hareli et al. [International Immunology (1999) 11: 635-641] further shows that GA-specific T-cells responses to in-vitro stimulation using Poly-YAK, Poly-YEA, Poly-YEK and Poly-EAK (copolymers comprising 3 of the 4 amino acids of GA) were heterogeneous responses with little or no cross reactivity (see Table 2, therein), thus substantiating copolymers comprising 3 of the 4 amino acids of GA cannot mimic GA.

The present inventors agree that 3 amino acids copolymers cannot mimic GA when used in the in-vitro stimulation step. However, a specific, reproducible and robust response to GA in-vitro stimulation was obtained when the present inventors counter intuitively used Poly-YAK in the in-vivo immunization step.

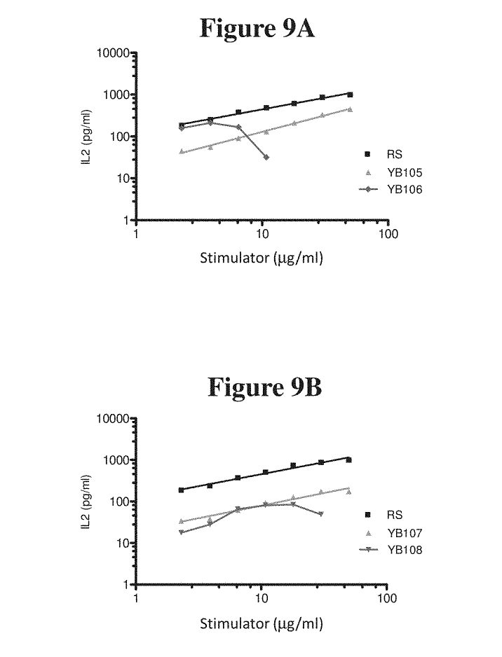

As is illustrated hereinunder and in the examples section, which follows, the present inventors show that in-vivo immunization with GA followed by in-vitro stimulation of extracted lymph node cells (LNCs) with either Poly-YAK, Poly-YEA, Poly-YEK or Poly-EAK did not result in in-vitro stimulation comparable to the in-vitro stimulation of the extracted LNCs with GA reference standard (RS) (Example 6, Table 8 and FIGS. 9A-B). Taken together, the results of using 3 amino acids copolymers as in-vitro stimulators in the GA potency assay were not reproducible nor robust, exhibiting no dose-response correlation.

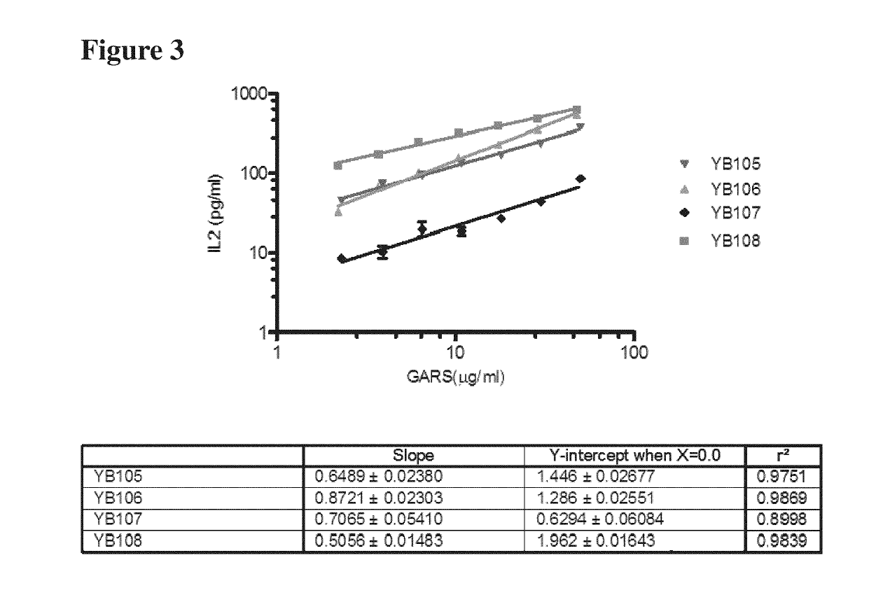

Following, the present inventors have uncovered that immunization of mice using the copolymers of 3 of the 4 amino acids comprising GA followed by in-vitro stimulation with GA resulted in specific, reproducible and robust IL-2 secretion to the medium that can be used to quantitatively determine the relative biological activity of GA. The inventors further demonstrate that according to the curve slope, linearity and intercept of the IL-2 secretion response, Poly-YAK is the preferred copolymer for the in-vivo immunization step (Example 3, FIG. 3). Moreover, the inventors present several cytokines secreted from the harvested LNCs in response to GA in-vitro stimulation (Example 5, Table 7).

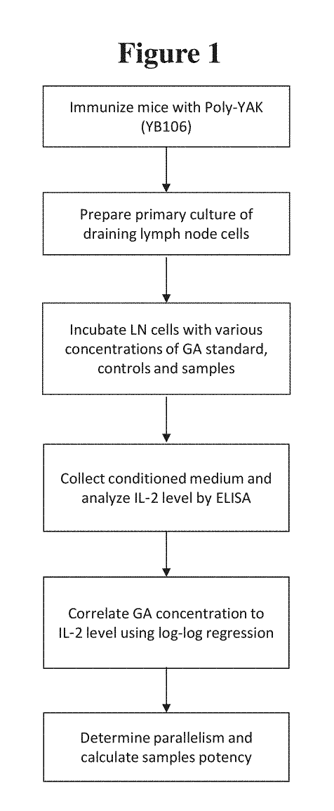

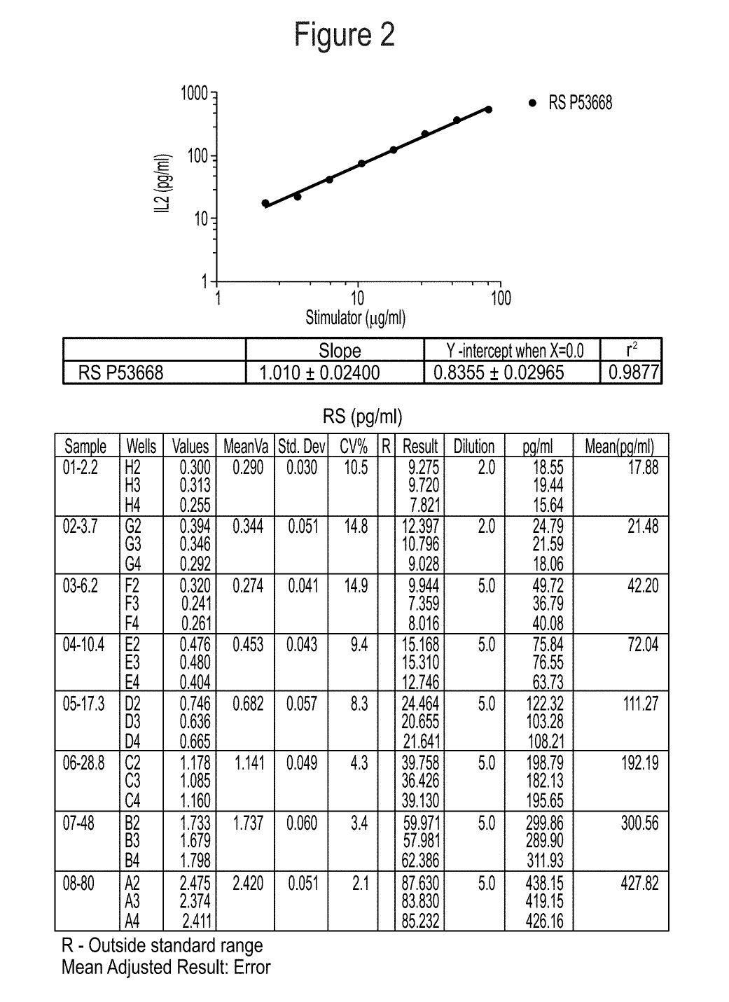

Consequently, the present teachings suggest a potency assay protocol (Example 2, FIGS. 1-2) comprising immunizing mice with Poly-YAK in CFA, stimulating extracted LNCs with different amounts of GA reference standard (RS) or tested batch of GA and determining the amount of IL-2 secreted by the cells into the culture media. Parallelism of the RS and tested batch curves and the relative potency of the tested batch are determined accordingly.

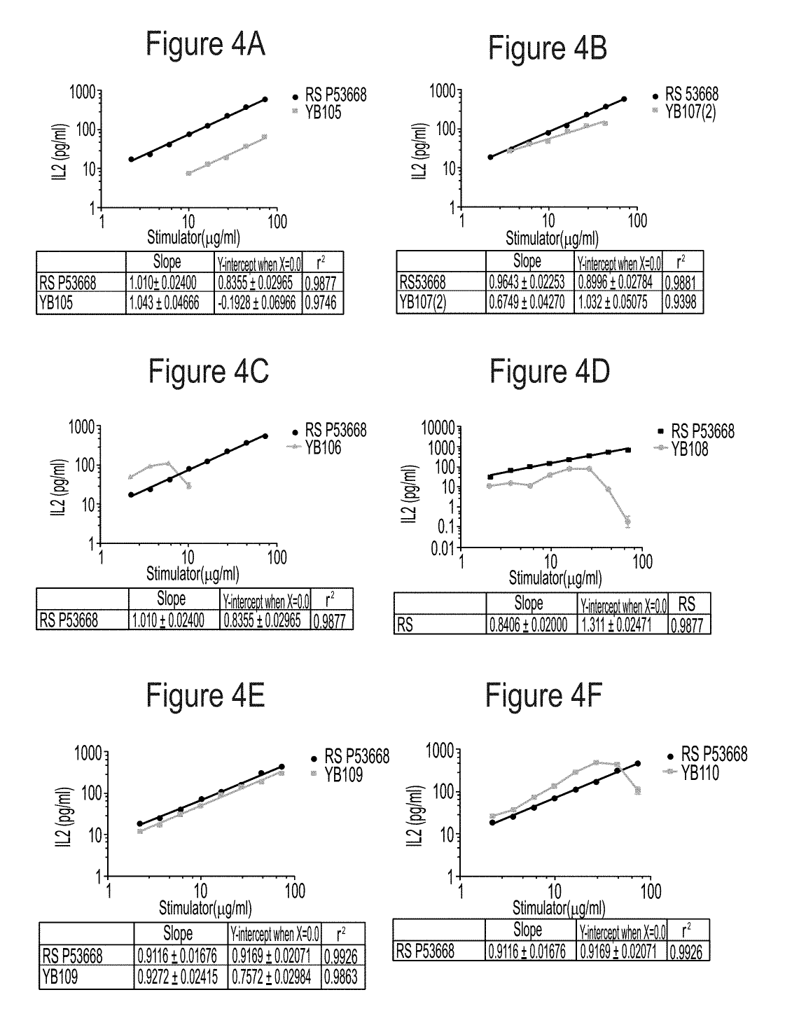

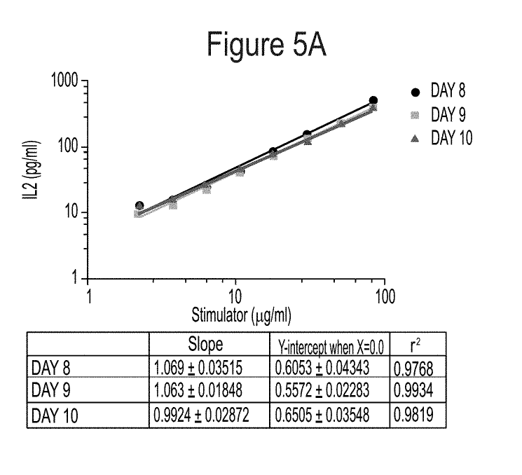

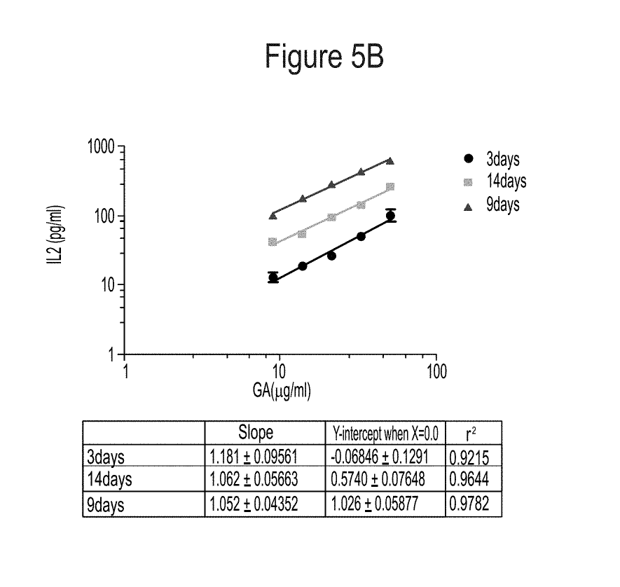

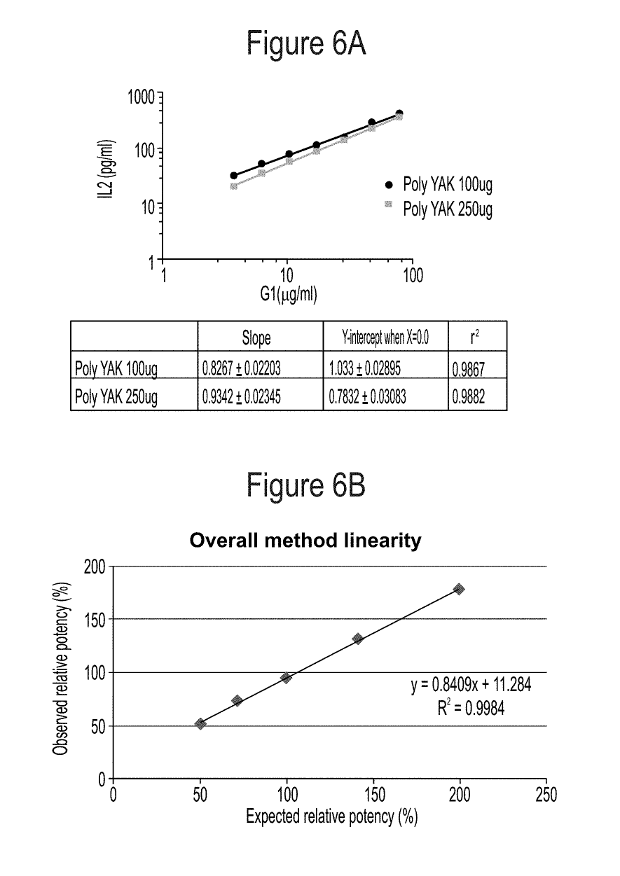

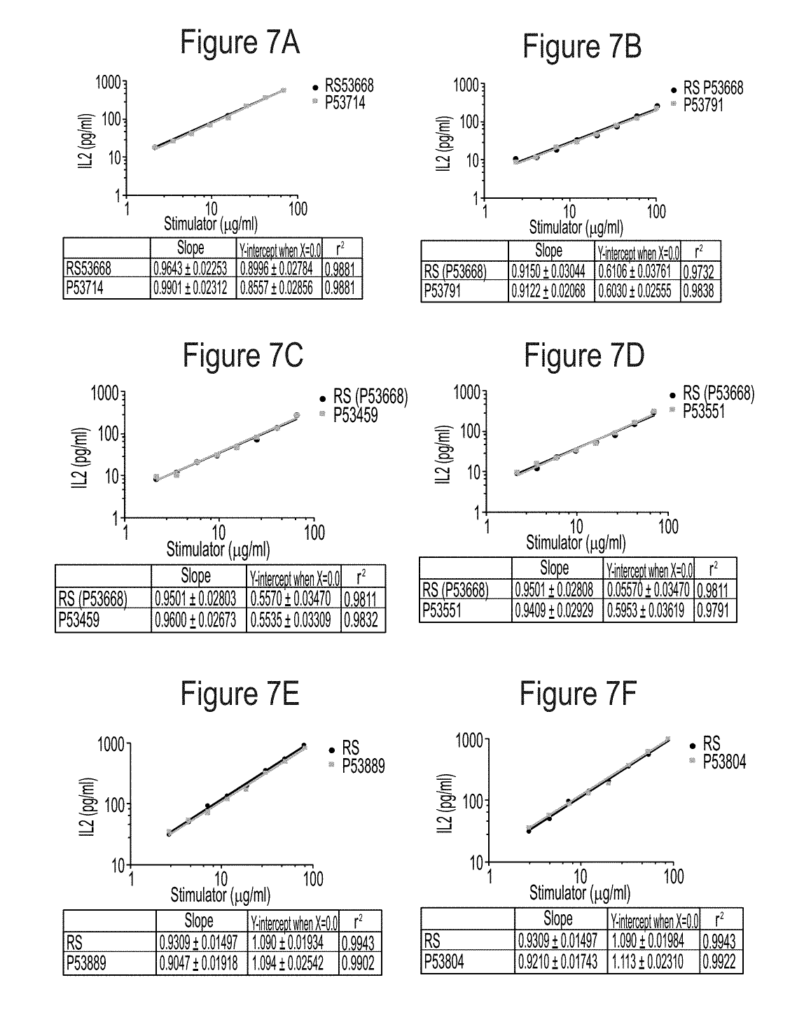

As is shown in Example 4, the ex-vivo potency assay developed by the present inventors demonstrated a strong dose-dependent in-vitro IL-2 secretion in response to GA. The assay was robust (FIGS. 5A, 5B and 6A and Table 4), accurate (FIG. 6B), highly specific to stimulation with GA (FIGS. 4A-F) and showed acceptable levels of relative potency of several COPAXONE® batches as well as GA non-commercial batches, passing the acceptance criteria of the assay (FIGS. 7A-F and 8A-F and tables 5-6).

Thus, according to a first aspect of the present invention, there is provided a method of determining relative potency of a batch of glatiramer acetate (GA), the method comprising:
(a) immunizing a mammal with an immunization effective amount of a Poly-YAK copolymer (pYAK) so as to obtain an immunized mammal;
(b) preparing a primary culture of T-cells from the immunized mammal;
(c) individually stimulating samples comprising substantially identical number of cells of the primary culture of T-cells with a predetermined amount of:
   (i) a reference standard (RS) batch of GA; or
   (ii) the batch of GA, wherein the predetermined amount of (i) is substantially identical to the predetermined amount of (ii);
(d) determining a stimulation parameter of the cells in each of the samples following a predetermined incubation time; and
(e) comparing the stimulation parameter in the cells of step (c)(i) with the stimulation parameter of the cells of step (c)(ii), so as to determine the relative potency of the batch of GA.

As used herein, the term “potency” refers to the measure of the biological activity of the product (i.e.; GA), based on the attribute of the product which is linked to the relevant biological properties (i.e.; T-cells specific stimulation).

As used herein, the term “relative potency” refers to a qualitative measure of potency of a batch of GA relatively to a standard reference (RS) of GA having a known potency.

According to specific embodiments the potency of a batch of GA is determined relatively to the known potency of a GA reference standard (RS).

As used herein, the phrase “GA reference standard” or “RS” refers to a standardized GA which is used as a measurement base for GA. RS provides a calibrated level of biological effect against which new preparations of GA can be compared to.

According to a specific embodiment, the reference standard is characterized by optimum potency and quality of an active component (i.e., GA) that is effective in treating the disease (e.g., multiple sclerosis).

According to specific embodiments RS is a COPAXONE® commercially available batch.

As used herein, the term “batch” refers to a specific quantity of a drug (i.e; GA) that is intended to have uniform character and quality, within specified limits, and is produced according to a single manufacturing order during the same cycle of manufacture.

As used herein, the term “glatiramer acetate” refers to a random copolymer composed of acetate salts of synthetic polypeptides, containing four naturally occurring amino acids, found in myelin basic protein, in a specified average molar fraction of L-glutamic acid (E): 0.129-0.153; L-alanine (A): 0.392-0.462; L-tyrosine (Y): 0.086-0.100; and L-lysine (K): 0.300-0.374. Chemically, GA is designated (PDR) L-glutamic acid polymer with L-alanine, L-lysine and L-tyrosine, acetate (salt) and its structural formula is:

(Glu,Ala,Lys,Tyr)ₓ.xCH₃COOH

(C₅H₉NO₄.C₃H₇NO₂.C₆H₁₄N₂O₂.C₉H₁₁NO₃)ₓ.xC₂H₄O₂

### CAS-147245-92-9

The average molecular weight of GA is 4,700-11,000 daltons [Physician's Desk Reference (PDR)]. Other average molecular weights for GA, lower than 40 kDa, are also encompassed by the present disclosure.

According to specific embodiments, the GA has an average molecular weight of about 4 to about 9 kDa, about 4 to about 8 kDa, about 5 to about 8 kDa, or about 6 to about 8 kDa.

According to specific embodiments, the GA contains less than 10%, less than 5%, or less than 2.5% of species of GA having a molecular weight of 40 kDa or more.

According to a specific embodiment the GA is substantially free of species of GA having a molecular weight of over 40 kDa (e.g., less than 0.1%).

According to a specific embodiment over 75% of the GA has a molar fraction within the molecular weight range from about 2 kDa to about 20 kDa.

As used herein, the term “copolymer” refers to a mixture of polypeptides containing specified amino acids having different molecular weights and sequences but consisting of only the specified amino acids. In the case of pYAK for example, the copolymer consists of only tyrosine, alanine and lysine in a randomized order.

According to a specific embodiment, GA is the drug manufactured under the trade name Copaxone™.

GA can be used for the treatment of multiple sclerosis e.g., relapsing remitting multiple sclerosis (RRMS), including patients who have experienced a first clinical episode and have MRI features consistent with multiple sclerosis. GA can also be used in the treatment of other autoimmune and non-autoimmune diseases, such as inflammatory bowel disease, hemolytic anemia, thyroiditis, Crohn's disease and non-autoimmune central nervous system (CNS) diseases (e.g.; Neural degeneration, Alzheimer's disease and Parkinson's disease).

Manufacturing procedures for producing GA are known in the art and have been described for example in U.S. Pat. Nos. 3,849,550; 5,800,808; 5,981,589; 6,048,898; 6,620,847; 7,049,399; 7,495,072; U.S. Patent Publication Nos. 2006/0154862; 2006/00172942 and 2007/0141663; and PCT Publication No. WO 00/05250, each of which is incorporated herein by reference.

Typically, the process for the synthesis of GA is based on the polymerization of N-carboxyanhydrides (NCA) of alanine, γ-benzyl glutamate, N-trifluoroacetyl lysine and tyrosine, in anhydrous dioxane using diethylamine as initiator. The deblocking of γ-benzyl groups (first deprotection) is effected by stirring the protected copolymer in hydrogen bromide in glacial acetic acid for 10-50 hours at room temperature. These conditions also facilitate the cleavage of the polypeptide so as to achieve the molecular weight of 4,700-11,000 daltons for GA. A test reaction is performed on every batch of GA at different time periods to determine the reaction time needed at a given temperature to achieve trifluoroacetyl polypeptides of a proper molecular weight profile.

The next step in the process is the removal of Nε-trifluoroacetyl groups (second deprotection) by treatment with a nitrogen base with weak basicity, such as 1 M piperidine.

In the final steps, GA is obtained by purification through dialysis, followed by treatment with acetic acid to form the acetate salt and by another purification by dialysis against water.

Alternative processes for preparing GA are also included. Thus, according to an exemplary embodiment, deblocking of the benzyl protecting groups is effected by palladium carbon.

According to an exemplary embodiment benzyl deprotection is effected by hydrogenolysis.

According to another exemplary embodiment NCA amino acids are used wherein the protecting group is selected from -methoxybenzyl and -benzyl.

GA with the required average molecular weight (see above, e.g.; 4.7 to 11 kDa) can be produced either by chromatography on a molecular weight sizing column or gel, and collection of the molecular weight ranges desired or by partial acid or enzymatic hydrolysis to remove the high molecular weight species with subsequent purification by dialysis or ultrafiltration. Further methods to produce GA having the required average molecular weight are based on the preparation of the desired species while the amino acids are still protected, followed by deprotection.

The term “Poly-YAK copolymer” (interchangeably referred to as Poly-YAK and pYAK) as used herein, refers to a copolymer containing tyrosine (Y), alanine (A), and lysine (K), in a molar fraction of about L-tyrosine (Y): 0.05-0.25; L-alanine (A): 0.3-0.6; and L-lysine (K): 0.1-0.5, with an average molecular weight of 2 KDa to 40 KDa.

According to specific embodiments Poly-YAK formula is:

{[AA₁]a₁-[AA₂]b₁-[AA₃]c₁}-{[AA₁]a₂-[AA₂]b₂-[AA₃]c₂} . . . {[AA₁]a₍ₙ₋₁₎-[AA₂]b₍ₙ₋₁₎-[AA₃]c₍ₙ₋₁₎}-{[AA₁]aₙ-[AA₂]bₙ-[AA₃]cₙ}

### Wherein AA₁ is L-tyrosine;

### AA₂ is L-alanine;

### AA₃ is L-lysine;

n represents the number of {[AA₁]a-[AA₂]b-[AA₃]c} units in the copolymer whereas each of a₁, a₂, . . . a₍ₙ₋₁₎ and an, each of b₁, b₂, . . . b₍ₙ₋₁₎ and bₙ and each of c₁, c₂, . . . c₍ₙ₋₁₎ and cₙ is independently 0 or a positive integer that ranges from 1 to 100, or from 1 to 80, or from 1 to 50, or from 1 to 30, or from 1 to 20, including any subranges between these ranges; such that a mole % of AA₁ ranges from 5 to 25; a mole % of AA₂ ranges from 30 to 60; and a mole % of AA₃ ranges from 10 to 50.

As used herein the term mole % of AA₁=(a₁+a₂+ . . . +a₍ₙ₋₁₎+aₙ)/the total number of residues in the copolymer×100;

the term mole % of AA₂=(b₁+b₂+ . . . +b₍ₙ₋₁₎+bₙ)/the total number of residues in the copolymer×100;

and the term mole % of AA₃=(c₁+c₂+ . . . +c₍ₙ₋₁₎+cₙ)/the total number of residues in the copolymer×100.

According to specific embodiments Poly-YAK molar ratios are about L-tyrosine (Y): 1; L-alanine (A): 5.2; and L-lysine (K): 3.7.

According to other specific embodiments Poly-YAK molar ratios are about L-tyrosine (Y): 1; L-alanine (A): 5.3; and L-lysine (K): 3.5.

According to specific embodiments the average molar fraction of the three amino acids L-alanine, L-tyrosine and L-lysine are about the same to the respective molar fraction in GA.

According to other specific embodiments the average molecular weight is between about 2 KDa to 40 KDa, between about 3 KDa to 35 KDa, or between about 5 KDa to 25 KDa.

According to specific embodiments Poly-YAK average molecular weight is 20 kDa.

According to other specific embodiments the average molecular weight of Poly-YAK is about the same to the average molecular weight of GA.

According to specific embodiments the process of Poly-YAK production is similar to the process of GA production as described in details hereinabove with the adjustments to contain only the amino acids L-tyrosine; L-alanine and L-lysine.

According to a specific embodiment the process of Poly-YAK production is performed according to the process described in Example 1. Briefly, polymerization of NCA-TFA-Lys, NCA-Ala and NCA-Tyr is effected in anhydrous dioxane using diethylamine as initiator. The deblocking of γ-benzyl groups (first deprotection) is effected by stirring the protected copolymer in hydrogen bromide in glacial acetic acid for about 20 hours at room temperature. The next step in the process is the removal of N^{ε}-trifluoroacetyl groups (second deprotection) by treatment with piperidine. In the final steps, Poly YAK is obtained by purification through dialysis, followed by treatment with acetic acid to form the acetate salt and by another purification by dialysis against water.

According to a specific embodiment the process of Poly-YAK production is performed according to the process described in Fridkis-Hereli et al. [International Immunology (1999) 11: 635-641].

Methods for characterization of copolymers are well known to the skilled in the art, for example by chromatographic methods. Non-limiting example for a chromatographic method is size exclusion high pressure liquid chromatography (SEC-HPLC). SEC-HPLC is a chromatographic method that separates molecules according to their size; large molecules elute from the column first, small molecules elute later.

According to specific embodiments, the apex retention time of Poly-YAK in SEC-HPLC is 24.13 minutes as assayed in Example 1 of the Examples section which follows.

As used herein, the term “immunizing” or “immunization” refers to administering antigen to a host mammal in order to induce the host immune system to respond specifically.

As used herein, the term “antigen” refers to a foreign substance that, when introduced into the body, is capable of stimulating an immune response, specifically activating lymphocytes. According to specific embodiments the antigen stimulates clonal expansion of antigen specific reactive T cells.

Accordingly, the immunizing antigen of the present invention is Poly-YAK.

As used herein, the phrase “an immunized mammal” refers to a mammal that we immunized with the antigen (i.e.; Poly-YAK).

The phrase “immunization effective amount” refers to the amount of the antigen resulting in production/clonal expansion of T cells that respond to GA in-vitro stimulation in a specific dose-response manner that can be quantitated using a stimulation parameter as further described hereinbelow.

Generally, following immunization, T cells of the immune system recognize immunogenic peptides presented by class I or class II major histocompatibility complex (MHC) molecules, expressed on antigen presenting cells (APC). The specificity of antigen recognition by T cells is defined by the affinity of the T cell receptor to the MHC-peptide complex as well as the primary sequence of the antigenic peptide. Following antigen recognition, clonal expansion of the specific T cells occurs. A second stimulus, causes differentiation and secretion of a variety of cytokines, which are typical of the expanded T-cell population. Proliferating helper T cells, which develop into effector T cells, differentiate into two major subtypes of cells known as Tₕ1 and Tₕ2 cells, each subtype interacts with a different cell partner and secretes a typical set of cytokines.

As mentioned, the present invention is based on effecting the first stimulus by immunizing a host mammal, as further described hereinbelow, in-vivo (also referred to as activation) with Poly-YAK, and the second stimulus by in-vitro stimulation (also referred to as stimulation) with GA.

According to specific embodiments the immunization effective amount of pYAK comprises 100 to 250 μg per mammal per bust.

Non limiting examples for mammals that can be used in the context of the present invention include male or female non-human mammal e.g., a rodent, a rabbit, a cat, a dog and a sheep.

According to specific embodiments the mammal is a rodent such as a mouse or a rat.

According to specific embodiments the rodent is a mouse.

According to specific embodiments the mouse is a female (SJLXBALB/C) F1 mouse.

According to specific embodiments the mouse is 8 to 12 weeks old.

Typically, antigen preparations for in-vivo immunization must be sterile and, ideally, isotonic, pH neutral, and free of urea, acetic acid, and other toxic solvents.

Immunization may be administered once or in multiple boosts, e.g., 2-3 boosts. Suitable routes of immunization depend on the host animal and may, for example, include footpad, tale base, intraperitoneal, intramuscular, intradermal, of subcutaneous injections.

According to specific embodiments the route of immunization is footpad injection.

The immunization active agent (Poly-YAK) may be administered without any adjuvant or it may be used in conjunction with an adjuvant such as by emulsifying the antigen with in an adjuvant suitable for pre-clinical use.

Thus, according to specific embodiments immunizing comprises administering an effective amount of an adjuvant simultaneously with the Poly-YAK copolymer (pYAK).

As used herein the term “adjuvant” refers to a component that potentiates the immune responses to an antigen and/or modulates it towards the desired immune responses. An immunologic adjuvant is defined as any substance that acts to accelerate, prolong, or enhance antigen-specific immune responses when used in combination with specific antigens. Non limiting examples of adjuvant that can be used include Complete Freund's adjuvant (CFA), Incomplete Freund's adjuvant (IFA), other microorganism-derived compounds [monophosphoryl lipid A (MPL, or the synthetic RC259), muramyl dipeptides and tripeptides (MDP and MTP), and TDM (trehalose dimycolate), etc.], other emulsions (TiterMax, Montanides, EMULSIGENS, Syntex Adjuvant Formulation (SAF), MF-59, and Specol, etc.), saponins (Quil A, and QS-21); aluminum compounds (e.g., alum), cytokines and immunostimulatory nucleic acids (e.g., CpG oligonucleotides), liposomes, virus-like particles, polymeric microspheres (polylactide co-glycolides), nanoparticles, subcutaneously-implanted chambers, or any other adjuvant that is found to be suitable for animal pre-clinical use.

According to specific embodiments the adjuvant is complete Freund's adjuvant (CFA).

According to specific embodiments one part or less of CFA is added to one part of Poly-YAK.

Following immunization a primary culture of T cells is prepared.

According to specific embodiments preparing a primary culture of T-cells is effected 3 to 14 days following immunizing the mammal.

The phrase “3 to 14 days following immunizing” refers to 3 to 14 days after the first boost of immunization.

According to other specific embodiments preparing a primary culture of T-cells is effected between 5 to 12 days; 7 to 11 days; 8 to 10 days; or 9 to 11 days following immunizing the mammal. According to a specific embodiment, preparing a primary culture of T-cells is effected between 8 to 10 days.

As used herein the term “primary culture” refers to non-immortalized, non-genetically modified cells directly removed from the immunized host mammal and their subsequent growth in a favorable controlled artificial environment in an appropriate cell density with an appropriate growth medium, temperature and gas mixture.

It will be appreciated that the present invention envisages generating cell lines from the immunized host mammal and their use.

As used herein T-cells or T-lymphocytes are CD3+ T-cells, which can be either CD4+ subtype or CD8+ subtype.

The primary culture of T cells comprises according to specific embodiments at least 10% T cells.

Methods for preparation of primary culture of T-cells are well known to the skilled in the art (see for example Matheu and Cahalan, (2007), J Vis Exp. 9: 409). For example, lymph nodes (LN) and/or spleen are harvested from a sacrificed mammal. Non liming examples for LNs that can be used include the popliteal, the axillary, the inguinal, the sacral or the lumbar LNs. Single cells suspensions are extracted by forcing the LNs and/or spleen through a cell strainer, collected, centrifuged and re-suspended to the required density. Optionally, separation of white blood cells can be obtained by separation on a Ficoll gradient or using erythrocytes lysis buffer (e.g.; ACK). Enrichment of specific cell populations can be obtained by methods well known in the art, for examples magnetic cell separation.

The T cells may be prepared from lymph nodes of the immunized animal. Alternatively the T cells are prepared from spleen of the immunized animal. Alternatively, the T cells are prepared from peripheral blood of the immunized animal.

Following preparation of primary culture of T-cells, predetermined amount of cells are incubated in tissue culture plates (e.g.; 12, 24, 96, 384 wells plates) with the appropriate Growth medium and stimulated with a predetermined amount of GA reference standard (RS) or a batch of GA. Selection of the medium is well within the capabilities of skilled in the art. Thus, for example, the medium can be a commercially available cell culture medium [e.g. DCCM-1 (can be obtained for example from Biological industries, Cat. No. 05-010-1A) and AIM-V (can be obtained for example from InVitrogen, Cat. No. 12055-091), RPMI (can be obtained for example from Sigma-Aldrich, Cat. No. R0883), and EX-CELL Hybridoma medium (can be obtained for example from Sigma-Aldrich, Cat. No H4281)] optionally, the medium is supplemented with L-glutamine, non-essential amino acids, sodium pyruvate, antibiotic/antimycotic solution, 2-mercaptoethanol and serum.

According to specific embodiments of the invention the medium does not contain serum.

Selection of the predetermined amount of cells incubated for in-vitro stimulation that will result in detectable T cell stimulation is well within the capabilities of the skilled in the art. Thus, for example cell concentration can be 1×10⁵/ml to 5×10⁷/ml; 1×10⁵/ml to 1×10⁷/ml; 5×10⁵/ml to 5×10⁷/ml; 1×10⁶/ml to 5×10⁷/ml; or 1×10⁶/ml to 1×10⁷/ml.

According to specific embodiments the cell concentration is 5×10⁶/ml.

Selection of the GA concentration used for in-vitro stimulation that will result in T cell stimulation is well within the capabilities of skilled in the art. Preferably, the GA concentration used should be within the linear range of the selected stimulation parameter. Thus, for example GA concentration can be 1 μg/ml to 25 μg/ml; 1 μg/ml to 50 μg/ml; 1 μg/ml to 100 μg/ml; 2 μg/ml to 80 μg/ml; 5 μg/ml to 80 μg/ml; 2 μg/ml to 60 μg/ml; or 5 μg/ml to 60 μg/ml. According to specific embodiments predetermined amount of the RS batch of GA and the batch of GA comprise 2 μg/ml to 80 μg/ml.

The number of tested GA stimulation concentrations can be at least 1, at least 2, at least 3, at least 5, at least 6, 1-10, 2-10, 3-10, 5-10, 1-5, 2-5 and 3-5 different concentrations.

The number of samples repeats for each of the tested GA concentrations can be 2, 3, 4, 5 or 6 repeats.

According to specific embodiments each of the samples stimulated with the predetermined amount of the RS batch of GA and the batch of GA comprise at least 2 repeats.

According to specific embodiments each of the samples stimulated with the predetermined amount of the RS batch of GA and the batch of GA comprise at least 3 repeats.

According to specific embodiments the assay may further include positive and negative control samples. The positive control for the assay may include agents inducing non-specific T cell activation/stimulation, for example Concavalin A (ConA, can be obtained for example from Sigma, Cat. No. C-5275) that induces mitogenic activity of T-lymphocytes in a non-specific manner.

Negative control for the assay may include agents which are not cross reactive with GA, for example Myelin Basic Protein (MBP) peptide 87-99 (can be obtained for example from BACHEM, Cat. No. H-1964).

As used herein, the term “stimulation parameter” refers to a quantitatively measurable T cell response which is indicative of T cell specific activation. Examples include, but are not limited to cytokine secretion, cell proliferation, cell survival, apoptosis, distribution of cell populations and expression of activation markers.

Specific methods of monitoring T cell stimulation are known in the art and include for example, cytokine secretion assays such as cytokine arrays (for example Mouse Cytokine Array Panel A, array kit—R&D Systems) and specific cytokines ELISAs (for example IL-2, IL-3, IL-4, IL-10, IL-13 and IFNγ, can be obtained from R&D Systems for example); survival assay such as the MTT test which is based on the selective ability of living cells to reduce the yellow salt MTT (3-(4,5-dimethylthiazolyl-2)-2,5-diphenyltetrazolium bromide) (can be obtained for example from Sigma, Aldrich) to a purple-blue insoluble formazan precipitate; proliferation assays such as the BrDu assay (for example Cell Proliferation ELISA BrdU colorimetric kit (Roche) and CFSE assay (can be obtained for example from eBioscience); Apoptosis assays such as the TUNEL assay (can be obtained for example from Roche); the Annexin V assay [for example ApoAlert® Annexin V Apoptosis Kit (Clontech Laboratories, Inc., CA, USA)]; evaluation of distribution of cell populations such as CD3+ T cells, CD4+ T cells, CD8+ T cells and B220+ T cells; activation marker evaluation such as IL-2R and CD62; as well as various RNA and protein detection methods (which detect level of expression and/or activity) such as ELISA, Western blot, RIA, FACS, immunohistochemistry, In-situ and in-vitro activity assays which are further described hereinbelow.

Enzyme Linked Immunosorbent Assay (ELISA):

This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western Blot:

This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabeled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Radio-Immunoassay (RIA):

In one version, this method involves precipitation of the desired protein (i.e., the substrate) with a specific antibody and radiolabeled antibody binding protein (e.g., protein A labeled with 1¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Fluorescence Activated Cell Sorting (FACS):

This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Immunohistochemical Analysis:

This method involves detection of a substrate in situ in fixed cells by substrate specific antibodies. The substrate specific antibodies may be enzyme linked or linked to fluorophores. Detection is by microscopy and subjective or automatic evaluation. If enzyme linked antibodies are employed, a colorimetric reaction may be required. It will be appreciated that immunohistochemistry is often followed by counterstaining of the cell nuclei using for example Hematoxyline or Giemsa stain.

In Situ Activity Assay:

According to this method, a chromogenic substrate is applied on the cells containing an active enzyme and the enzyme catalyzes a reaction in which the substrate is decomposed to produce a chromogenic product visible by a light or a fluorescent microscope.

In Vitro Activity Assays:

In these methods the activity of a particular enzyme is measured in a protein mixture extracted from the cells. The activity can be measured in a spectrophotometer well using colorimetric methods or can be measured in a non-denaturing acrylamide gel (i.e., activity gel). Following electrophoresis the gel is soaked in a solution containing a substrate and colorimetric reagents. The resulting stained band corresponds to the enzymatic activity of the protein of interest. If well calibrated and within the linear range of response, the amount of enzyme present in the sample is proportional to the amount of color produced. An enzyme standard is generally employed to improve quantitative accuracy.

According to specific embodiments the stimulation parameter is cytokine secretion.

As used herein, the term “cytokine” refers to a family of small secreted regulatory proteins, such as the interleukins and lymphokines, which are released by cells of the immune system and act as intercellular mediators in the generation of an immune response.

Non limiting examples for cytokines that can be used according to the teachings of the present invention include IFNg, GM-CSF, IL-2, IL-3, IL-4, IL-6, IL-10, IL-13, I-309, IP-10 and MIP-1α.

According to specific embodiments the cytokine is an interleukin.

As used herein, the term “interleukin” (IL) refers to a family of small secreted proteins that mediate communication between cells (namely, regulate cell growth, differentiation and motility) and are particularly important in promoting the growth, differentiation and activation of white blood cells thus involved in stimulating immune responses.

According to specific embodiments the interleukins are Tₕ1 related interleukins.

According to specific embodiments the interleukins are Tₕ2 related interleukins.

According to other specific embodiments the interleukins are Tₕ0 related interleukins.

Non limiting examples for interleukins that can be used with the teaching of the present invention include IL-2, IL-3, IL-4, IL-6, IL-10 and IL-13.

According to a specific embodiment the interleukin is interleukin-2 (IL-2).

IL-2 is produced by T cells in response to antigenic or mitogenic stimulation, acting to regulate the immune response. It stimulates the proliferation of T cells and the synthesis of other T cell-derived cytokines, stimulates the growth and cytolytic function of NK cells to produce lymphokine-activated killer cells, is a growth factor for and stimulates antibody synthesis in B cells, and may promote apoptosis in antigen-activated T cells.

The level of IL-2 secreted by the cells into the media can be determined for example using an IL-2 specific ELISA (such as mouse IL2 ELISA, BD OptEIA™, Cat. No. 55148).

The incubation time may vary and may depend on the stimulation parameter being determined. According to a specific embodiment, the incubation time is between 12 hours to 48 hours. According to some embodiments of the invention, the incubation time is between 19 to 25 hours; 21 to 25 hours; 19 to 23 hours; or 21 to 23 hours. According to specific embodiments of the invention, the incubation time is 17 to 27 hours.

As mentioned, the last phase of the method comprises comparing the stimulation parameter in the cells stimulated with the batch of GA with the stimulation parameter in the cells stimulated with RS, so as to determine the relative potency of the batch of GA.

According to specific embodiments the method of determining relative potency of a batch of glatiramer acetate (GA) further comprising generating stimulation curves of the samples incubated with predetermined amount of the RS batch of GA and of the samples incubated with predetermined amount of the batch of GA, so as to determine parallelism. The stimulation curves can be generated manually, automatically, or a combination thereof. In such curves the concentration of the stimulator is typically placed on the x axis while the stimulation intensity is typically placed on the y axis.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

As used herein, the term “parallelism” refers to a mean of quantitative determination of how equal is the stimulation induced by the batch of GA to that induced by the RS. Parallelism can be established if a horizontal shift of the curve of the stimulation induced by the batch of GA places the curve on top of the curve of the stimulation induced by the RS. When analyzing linear curves, parallelism can be established if the two lines have the same slope. According to specific embodiments parallelism of the RS stimulated curve and the batch of GA stimulated curve is determined with 80%, 85%, 90%, 95% or 100% confidence.

The present teachings can be used in the QA of the manufacturing procedures of GA for assessing the biological activity of GA as part of GA batch qualification.

Thus, according to another aspect of the present invention, there is provided a method of preparing a batch of glatiramer acetate as acceptable for pharmaceutical use, the method comprising:

(a) preparing a batch of GA;
(b) measuring the relative potency of the batch according to the methods described hereinabove; and
(c) qualifying the batch as acceptable for pharmaceutical use if the relative potency so measured in step (b) is between 80% and 125% of the RS batch of GA.

According to another aspect of the present invention, there is provided a method of preparing a pharmaceutical composition comprising GA, the method comprising:

(a) preparing a batch of GA;
(b) measuring the relative potency of the batch according to the according to the methods described hereinabove; and
(c) preparing the pharmaceutical composition with the batch of GA if the relative potency so measured in step (b) is between 80% and 125% of the RS batch of GA.

As used herein, the phrase “acceptable for pharmaceutical use” means approved by a regulatory agency of the Federal or a state government or listed in the U.S. Pharmacopeia or other generally recognized pharmacopeia for use in animals, and more particularly in humans.

Calculating relative potency and parallelism analysis are known in the art.

According to specific embodiments, the relative potency and parallelism are calculated as described in U.S. Patent Publication No. US20110189706 which is incorporated herein by reference.

According to specific embodiments the relative potency of a batch of GA is calculated according to:

% Potency=100*10̂{((YaveSa−YaveRS)/β*−(XaveSa−XaveRS)}, wherein

YaveSa is the average Log [stimulation parameter] of all tested sample points,
YaveRS is the average Log [stimulation parameter] of all RS points,
XaveSa is the average Log [stimulator] of all tested sample points,
XaveRS is the average Log [stimulator] of all RS points,
β* is the RS batch of GA and batch of GA combined slope, calculated according to:

β*=Σ(Y−Yave)*(X−Xave)/Σ(X−Xave); wherein

Y—Log [stimulation parameter],
X—Log [stimulator] is for each data point.
Yave, Xave—Y average, X average for all data points.

According to specific embodiments the relative potency of a batch of GA is calculated using a software suitable for biological assays, such as parallel line analysis software e.g., PLA (Stegmann Systems GmbH) and Gen5 data analysis software (BioTek).

The pharmaceutical composition with the batch of GA is prepared if the relative potency so measured is between 80% and 125% of the RS batch of GA.

According to specific embodiments the batch of GA is qualified for use when the relative potency so measured is between 80%-125%; 80% and 120%; 85% and 125%; 85% and 120%; 85% and 115%; 90% and 120%; 90% and 115%; 90% and 110%; 95% and 110%; 95% and 105%; 98% and 105%; or 98% and 102%.

According to another aspect of the present invention, there is provided a method of treating multiple sclerosis (MS) in a subject in need thereof, the method comprising injecting into the subject a pharmaceutical composition which comprises GA generated according to the method described hereinabove, thereby treating MS in a subject.

As disclosed herein, GA may be used as a sole therapy or in combination with one or more drugs for the treatment of MS.

As used herein the term “Multiple Sclerosis” (MS) refers to a chronic, debilitating inflammatory disease of the central nervous system (CNS) of autoimmune origin. MS is characterized by focal demyelination, loss of oligodendrocytes, and astrocytic scar formation in advanced stages of the disease. Histopathologically, acute inflammatory lesions are characterized by infiltrating lymphocytes and macrophages scattered throughout the periventricular white matter, spinal cord, brainstem and optic nerves. In the later stages of the disease, vascular infiltrates are less prominent, and loss of myelin and oligodendrocytes predominates. Eventually, myelin breakdown as the hallmark of the disease is brought about by the combined effects of autoantibodies against myelin proteins, complement activation, cytotoxic cells and cytokine-induced toxicity. MS disease activity can be monitored by magnetic resonance imaging (MRI) of the brain, accumulation of disability, as well as rate and severity of relapses.

There are five distinct disease stages and/or major types of MS.

Benign Multiple Sclerosis:

Benign multiple sclerosis is a retrospective diagnosis which is characterized by 1-2 relapses with complete recovery, no lasting disability and no disease progression for 10-15 years after the initial onset. Benign multiple sclerosis may, however, progress into other forms of multiple sclerosis.

Relapsing-Remitting Multiple Sclerosis (RRMS):

Patients suffering from RRMS experience sporadic relapses, as well as periods of remission. Lesions and evidence of axonal loss may or may not be visible on MRI for patients with RRMS.

Secondary Progressive Multiple Sclerosis (SPMS):

SPMS may evolve from RRMS. Patients afflicted with SPMS have relapses, a diminishing degree of recovery during remissions, less frequent remissions and more pronounced neurological deficits than RRMS patients. Enlarged ventricles, which are markers for atrophy of the corpus callosum, midline center and spinal cord, are visible on MRI of patients with SPMS.

Primary Progressive Multiple Sclerosis (PPMS):

PPMS is characterized by a steady progression of increasing neurological deficits without distinct attacks or remissions. Cerebral lesions, diffuse spinal cord damage and evidence of axonal loss are evident on the MRI of patients with PPMS.

Progressive-Relapsing Multiple Sclerosis (PRMS):

PRMS has periods of acute relapses while proceeding along a course of increasing neurological deficits without remissions. Lesions are evident on MRI of patients suffering from PRMS.

The relapsing forms of multiple sclerosis include: Relapsing-remitting MS (RRMS), Secondary Progressive MS (SPMS) and Progressive-relapsing MS (PRMS).

As used herein, the term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

As used herein, the term “subject” includes mammals, preferably human beings at any age which suffer from the pathology. Preferably, this term encompasses individuals who are at risk to develop the pathology.

According to specific embodiments, treating comprises reducing the frequency of relapses, reducing the mean cumulative number of Gd-enhancing lesions in the brain of the patient, reducing the mean number of new T₂ lesions in the brain of the patient, reducing the cumulative number of enhancing lesions on T₁-weighted images in the patient, reducing brain atrophy in the patient, increasing the time to a confirmed relapse in the patient, reducing the total number of confirmed relapses in the patient, reducing the progression of MRI-monitored disease activity in the patient, reducing total volume of T₂ lesions in the patient, reducing the number of new hypointense lesions on enhanced T₁ scans in the patient, reducing the total volume of hypointense lesions on enhanced T₁ scans in the patient, reducing the level of disability as measured by EDSS Score in the patient, reducing the change in EDSS Score in the patient, reducing the change in Ambulation Index in the patient, reducing the level of disability as measured by EuroQoL (EQ5D) questionnaire in the patient, and/or reducing the level of disability as measured by the work productivity and activities impairment General Health (WPAI-GH) questionnaire in the patient.

A 20 mg/day subcutaneous dose of GA has been shown to reduce the total number of enhancing lesions in MS patients as measured by MRI (G. Comi et al., Ann. Neurol. 49:290-297 (2001)).

As used herein, the phrase “Gd-enhancing lesions”, refers to lesions that result from a breakdown of the blood-brain barrier, which appear in contrast studies using gandolinium contrast agents. Gandolinium enhancement provides information as to the age of a lesion, as Gd-enhancing lesions typically occur within a six week period of lesion formation.

As used herein, the phrase “T₁-weighted MRI images” refers to an MRI-image that emphasizes T₁ contrast by which lesions may be visualized. Abnormal areas in a T₁-weighted MRI image are “hypointense” and appear as dark spots. These spots are generally older lesions.

As used herein, the phrase “T₂-weighted MRI image”, refers to an MR-image that emphasizes T₂ contrast by which lesions may be visualized. T₂ lesions represent new inflammatory activity.

Clinical evidence of a lesion is defined as signs of neurological dysfunction demonstrable by neurological examination. An abnormal sign constitutes clinical evidence even if no longer present, but was recorded in the past by a competent examiner.

Para-clinical evidence of a lesion is defined as the demonstration by means of various tests and procedures of the existence of a lesion of the CNS that has not produced clinical signs but that may or may not have caused symptoms in the past.

Such evidence may be derived from the hot-bath test, evoked response studies, neuroimaging, and expert neurological assessment. These tests are considered to be extensions of the neurological examination and not laboratory procedures.

As used herein, a patient at risk of developing MS (i.e. clinically definite MS) is a patient presenting any of the known risk factors for MS. The known risk factors for MS include anyone of a clinically isolated syndrome (CIS), a single attack suggestive of MS without a lesion, the presence of a lesion (in any of the CNS, PNS, or myelin sheath) without a clinical attack, environmental factors (geographical location, climate, diet, toxins, sunlight), genetics (variation of genes encoding HLA-DRB 1, IL7R-alpha and IL2R-alpha), and immunological components (viral infection such as by Epstein-Barr virus, high avidity CD4+ T cells, CD8+ T cells, anti-NF-L, antiCSF114(Glc)).

A clinically isolated syndrome (CIS) is a single clinical attack compatible suggestive of MS, such as optic neuritis, brain stem symptoms, and partial myelitis; and at least one lesion suggestive of MS.

Thus, the GA of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the GA accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, buccal, rectal, transmucosal, especially transnasal, intestinal or parenteral delivery, including intramuscular, subcutaneous, transdermal and intramedullary injections as well as intrathecal, direct intraventricular, intracardiac, e.g., into the right or left ventricular cavity, into the common coronary artery, intravenous, intraperitoneal, intranasal, or intraocular injections.

According to specific embodiments the routes of administration are oral and subcutaneous injections.

According to other specific embodiments the route of administration is subcutaneous injections.

According to a specific embodiment the pharmaceutical composition is a clear, colorless to slightly yellow, sterile, nonpyrogenic solution.

According to a specific embodiment each 1 ml of solution contains 20 mg of GA and 40 mg of mannitol.

According to another specific embodiment the pH range of the solution is approximately 5.5 to 7.0.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

The pharmaceutical composition described herein may be formulated for parenteral administration, e.g., by bolus injection or continues infusion. Formulations for injection may be presented in unit dosage form, e.g., in ampoules or in multi-dose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

Pharmaceutical compositions suitable for use in context of some embodiments of the invention include compositions wherein the active ingredients are contained in an amount effective to achieve the intended purpose. More specifically, a therapeutically effective amount means an amount of active ingredients (GA) effective to prevent, alleviate or ameliorate symptoms of a disorder (e.g., MS) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

According to specific embodiment, the dose of GA administered ranges from about 0.1 mg to about 1000 mg, from about 1 mg to about 100 mg, from about 5 mg to about 50 mg, or from about 10 mg to about 30 mg.

According to specific embodiment, the dose of GA administered is about 20 mg.

According to another specific embodiment, the dose of GA administered is about 40 mg.

According to specific embodiments, GA is administered at a frequency of about once every 30 days to about once every day, or at a frequency of about once every 7 days to about once every day.

According to other specific embodiments the dose of GA is administered at a frequency of about once every day.

According to other specific embodiment, the dose of GA is administered at a frequency of about 3 times over a period of seven days.

According to another embodiments, the GA is administered as part of a therapeutic regimen during which a cytokine antagonist or is also administered to the subject.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

According to specific embodiment, the pharmaceutical composition is in a prefilled syringe for self administration by the subject.

According to specific embodiments the method of preparing a batch of glatiramer acetate as acceptable for pharmaceutical use further comprising generating stimulation curves of the samples incubated with predetermined amount of the RS batch of GA and of the samples incubated with predetermined amount of the batch of GA, and qualifying the batch as acceptable for pharmaceutical use if the stimulation curves are parallel.

According to specific embodiments the method of preparing a pharmaceutical composition comprising GA further comprising generating stimulation curves of the samples incubated with predetermined amount of the RS batch of GA and of the samples incubated with predetermined amount of the batch of GA, and preparing the pharmaceutical if the stimulation curves are parallel.

As used herein, the term “room temperature” should be understood to mean a temperature ranging from about 20° C. to about 26° C.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Copolymers Synthesis

### Synthesis of YB-105 (Poly-EAK)

Step 1:

0.25 liter of anhydrous dioxane were added to a 0.5 liter round bottomed flask, while stirring, followed by addition of 5.00 g NCA-TFA-Lys, 3.01 g NCA-Ala, 2.10 g NCA-benzyl Glu consecutively. Following 10 min, when the solution was clear, 0.056 gr (0.55%) of diethylamine were added to initiate polymerization. The mixture turned cloudy after 5-10 minutes. The mixture was stirred at room temperature (20-24° C.) for 18 hours. After 18 hours, the reaction mixture was gradually added (during 1 minute) while stirring, to 0.75 liter of cold water at 15° C., leading to precipitation of white solid lumps. The product was separated by filtration through Buchner and washed with 1 liter of water. The product was dried under reduced pressure in a vacuum oven at 50° C. to yield 6.7 g of a white solid. The dry solid was grinded (intermediate I).

Step 2:

A 19% solution of HBr in AcOH was prepared by adding 0.11 liter of a solution of 33% hydrogen bromide in acetic acid to 0.11 liter of AcOH. The resulting solution was added at room temperature to the grinded intermediate from step I while stirring at ˜200 rpm. After 10 minutes the stirring was reduced to 100 rpm and continued for 18 hours at room temperature followed by stirring at 250 rpm for 2 hours. Most of the solid (80-90%) dissolved after 30 minutes and the entire solid dissolved after less than 20 hours. The clear solution was gradually added (during 1 minute), to 0.75 liter of water at 10° C., while stirring leading to the formation of white precipitate. The product was separated by filtration using Buchner and washed with water (intermediate II).

Step 3:

Intermediate II was added to 0.22 liter of water in 0.5 round bottomed flask. 25 ml piperidine was added to the stirred mixture. The mixture was stirred at room temperature for 16 hours and then filtered through a filter paper. The paper was washed with water to produce 0.3 liter of solution.

Step 4:

The crude product in solution was purified by Ultrafiltration/diafiltration using two 0.093 m² suspended screen PES Pall cassettes, cutoff 5K. The reaction solution was concentrated to 0.2 liter and diafiltration at transmembrane pressure (TMP) 2.0 bar, feed flow 150 liter/hour, was performed against 1.2 liters of water followed by 0.6 liters of 0.3% AcOH solution and finally with 0.6 liters of water. The purified solution was freeze dried resulting in 2.5 g of solid.

### Synthesis of YB-106 (Poly-YAK)

Step 1:

0.25 liter of anhydrous dioxane were added to a 0.5 liter round bottomed flask, while stirring, followed by addition of 5.00 g NCA-TFA-Lys, 3.01 g NCA-Ala and 1.08 g NCA-Tyr consecutively. Following 10 min, when the solution was clear, 0.05 gr (0.55%) of diethylamine were added to initiate polymerization. The mixture turned cloudy after 5-10 minutes. The mixture was stirred at room temperature (20-24° C.) for 20 hours. After 20 hours, the reaction mixture was gradually added (during 1 minute) while stirring, to 0.75 liter of cold water at 15° C., leading to precipitation of white solid lumps. The product was separated by filtration through Buchner and washed with 1 liter of water. The product was dried under reduced pressure in a vacuum oven at 50° C. to yield 6.04 g of a white solid. The dry solid was grinded giving 6.08 g solid (intermediate I).

Step 2:

A 19% solution of HBr in AcOH was prepared by adding 0.11 liter of a solution of 33% hydrogen bromide in acetic acid to 0.11 liter of AcOH. The resulting solution was added to the grinded intermediate from step I while stirring at ˜200 rpm. After 10 minutes the stirring was reduced to 100 rpm and continued for 20 hours at room temperature. The clear solution was gradually added (during 1 minute), to 0.75 liter of water at 10° C., while stirring leading to the formation of white precipitate. The product was separated by filtration using Buchner and washed with 0.7 liter of water. The product was dried under reduced pressure at 25° C. in a vacuum oven, giving rise to a white solid (intermediate II).

Step 3:

Intermediate II was added to 0.22 liter of water in 0.5 round bottomed flask. Following 0.5 hour of stirring 25 ml piperidine was added to the stirred mixture. After stirring for additional 1.5 hours, additional 25 ml piperidine was added in order to completely dissolve the remaining suspended solids. The mixture was stirred at room temperature for 18 hours. The mixture was then filtered through a filter paper. The paper was washed with water to produce 0.25 liter of clear and colorless solution. Water was added up to 0.5 liter through the filter resulting in ˜6.6% piperidine solution.

Step 4:

The crude product in solution was purified by Ultrafiltration/diafiltration using two 0.093 m² suspended screen PES Pall cassettes, cutoff 5K. The reaction solution was concentrated to 0.2 liter and diafiltration at transmembrane pressure (TMP) 2 bar, feed flow 100 liter/hour, was performed against 1.2 liters of water followed by 0.6 liters of 0.3% AcOH solution and finally with 0.6 liters of water. The purified solution was freeze dried resulting in 2.5 g of white to off white solid.

### Synthesis of YB-107 (Poly-YEA)

Step 1:

0.25 liter of anhydrous dioxane were added to a 0.5 liter round bottomed flask, while stirring, followed by addition of 3.01 g NCA-Ala, 2.1 g of NCA-benzyl-Glu and 1.62 gr of NCA-Tyr consecutively. Following 10 min, when the solution was clear, 0.056 gr (0.55%) of diethylamine were added to initiate polymerization. The mixture turned cloudy after 5-10 minutes. After 1-2 hours the mixture became very viscous thus additional 0.25 liter of anhydrous dioxane were added. The mixture was stirred at room temperature (20-24° C.) for 20 hours. After 20 hours, the reaction mixture was gradually added (during 1 minute) while stirring, to 1.5 liters of cold water at 15° C., leading to precipitation of white solid lumps. The product was separated by filtration through Buchner and washed with 1 liter of water. The product was dried under reduced pressure in a vacuum oven at 50° C. to yield 6.36 g of a white solid. The dry solid was grinded (intermediate I).

Step 2:

A 19% solution of HBr in AcOH was prepared by adding 0.11 liter of a solution of 33% hydrogen bromide in acetic acid to 0.11 liter of AcOH. 0.14 liter of the resulting solution was added to 6 g of the grinded intermediate from step I while stirring at ˜300 rpm. The mixture was stirred for 20 hours at 26° C. All solid dissolved after 4 hours. The clear solution was gradually added (during 1 minute), to 0.75 liter of water at 10° C., while stirring leading to the formation of white precipitate. The product was separated by filtration using Buchner and washed with 0.8 liter of water. The product was dried under reduced pressure at 25° C. in a vacuum oven, giving rise to a light brown solid (intermediate II).

Step 3:

Intermediate II was added to 0.22 liter of water in 0.5 round bottomed flask. Following 0.5 hour of stirring 25 ml piperidine was added to the stirred mixture. After stirring for additional 1.5 hours the colourless mixture became clear. The mixture was stirred at 200-400 rpm at room temperature for 21 hours. The mixture was then filtered through a filter paper. The paper was washed with water to produce 0.35 liter of clear and colorless solution.

Step 4:

The crude product in solution was purified by Ultrafiltration/diafiltration using two 0.093 m² suspended screen PES Pall cassettes, cutoff 5K. The reaction solution was concentrated to 0.2 liter and diafiltration at transmembrane pressure (TMP) 2 bar, feed flow 100 liter/hour, was performed against 1.2 liters of water followed by 0.6 liters of 0.3% AcOH solution and finally with 0.6 liters of water. The product precipitated at pH 4.7 after storage at 4° C. thus the pH of the solution was adjusted to 5.8 by addition of 20 ml of 1 M sodium acetate, leading to formation clear solution. The purified solution was freeze dried resulting in 1.17 g of solid.

### Synthesis of YB-108 (Poly-YEK)

Step 1:

0.25 liter of anhydrous dioxane were added to a 0.5 liter round bottomed flask, while stirring, followed by addition of 5.00 g NCA-TFA-Lys, 2.10 g of NCA-benzyl-Glu and 1.62 gr of NCA-Tyr consecutively. Following 10 min, when the solution was clear, 0.067 gr (0.55%) of diethylamine were added to initiate polymerization. The mixture was stirred at room temperature (20-24° C.) for 24 hours. After 24 hours, the reaction mixture was gradually added (during 1 minute) while stirring, to 0.75 liter of cold water at 15° C., leading to precipitation of white solid. The product was separated by filtration through Buchner and washed with 0.1-0.3 liter of water. The product was dried under reduced pressure in a vacuum oven at 50° C. to yield 6.23 g of a white solid. The dry solid was grinded (intermediate I).

Step 2:

A 19% solution of HBr in AcOH was prepared by adding 0.11 liter of a solution of 33% hydrogen bromide in acetic acid to 0.11 liter of AcOH. The resulting solution was added to 6 g of the grinded intermediate from step I while stirring at ˜200 rpm. After 10 minutes the stirring was reduced to 100 rpm and continued for 20 hours at room temperature. The clear solution was gradually added (during 1 minute), to 0.75 liter of water at 10° C., while stirring leading to the formation of white precipitate. The product was separated by filtration using Buchner and washed with 0.7 liter of water. The product was dried under reduced pressure at 25° C. in a vacuum oven, giving rise to 7.0 g of white solid (intermediate II).

Step 3:

Intermediate II was added to 0.22 liter of water in 0.5 round bottomed flask. Following 0.5 hour of stirring 25 ml piperidine was added to the stirred mixture. The mixture was stirred at 200-400 rpm at room temperature for 20 hours. The mixture was then filtered through a filter paper. The paper was washed with water to produce 0.35 liter of clear and colorless solution.

Step 4:

The crude product in solution was purified by Ultrafiltration/diafiltration using two 0.093 m² suspended screen PES Pall cassettes, cutoff 5K. The reaction solution was concentrated to 0.2 liter and diafiltration at transmembrane pressure (TMP) 2 bar, feed flow 150 liter/hour, was performed against 1.2 liters of water followed by 0.6 liters of 0.3% AcOH solution and finally with 0.6 liters of water. The purified solution was freeze dried resulting in 2.2 g of solid.

### Synthesis of YB-109 (short Poly-YEAK)

Step 1:

0.25 liter of anhydrous dioxane were added to a 0.5 liter round bottomed flask, while stirring, followed by addition of 5.00 gr NCA-TFA-Lys, 3.01 gr NCA-Ala, 2.10 gr NCA-benzyl-Glu and 1.08 NCA-Try consecutively. Following 10 min, when the solution was clear, 1.12 gr (10%) of diethylamine were added to initiate polymerization. The mixture turned cloudy after 5-10 minutes. The mixture was stirred at room temperature (20-24° C.) for 19 hours. After 19 hours, the reaction mixture was gradually added (during 1 minute) while stirring, to 0.75 liter of cold water at 15° C., leading to precipitation of white solid lumps. The product was separated by filtration through Buchner and washed with 1 liter of water. The product was dried under reduced pressure in a vacuum oven at 50° C. to yield 8.4 g of a white solid. The dry solid was grinded (intermediate I).

Step 2:

A 19% solution of HBr in AcOH was prepared by adding 0.11 liter of a solution of 33% hydrogen bromide in acetic acid to 0.11 liter of AcOH. The resulting solution was added to 3.4 g of the grinded intermediate from step I while stirring at ˜200 rpm. The mixture was stirred at 200 rpm for 24 hours at room temperature. The clear solution was gradually added (during 1 minute), to 0.75 liter of water at 10° C., while stirring leading to the formation of white precipitate. The product was separated by filtration using Buchner and washed with 0.7 liter of water (intermediate II).

Step 3:

The crude wet Intermediate II was added to 0.22 liter of water in 0.5 round bottomed flask followed by addition of 25 ml piperidine. The mixture was stirred at room temperature for 5 hours. The mixture became clear after less than 1 hour of stirring. The mixture was then filtered through a filter paper. The paper was washed with 50 ml of water to produce clear and colorless solution. Water was added up to 0.35 liter through the filter resulting in ˜6.6% piperidine solution.

Step 4:

The crude product in solution was purified by Ultrafiltration/diafiltration using two 0.093 m² suspended screen PES Pall cassettes, cutoff 5K. The reaction solution was concentrated to 0.2 liter and diafiltration at transmembrane pressure (TMP) 2 bar, feed flow 150 liter/hour, was performed against 1.2 liters of water followed by 0.6 liters of 0.3% AcOH solution and finally with 0.6 liters of water. The purified solution was freeze dried resulting in 0.55 g of solid with an average molecular weight of 4,962 Dal.

### Synthesis of YB-110 (long Poly-YEAK)

Step 1:

0.25 liter of anhydrous dioxane were added to a 0.5 liter round bottomed flask, while stirring, followed by addition of 5.00 gr NCA-TFA-Lys, 3.01 gr NCA-Ala, 2.10 gr NCA-benzyl-Glu and 1.08 gr NCA-Tyr, consecutively. Following 10 min, when the solution was clear, 0.01 gr (0.1%) of diethylamine were added to initiate polymerization. The mixture turned cloudy after 5-10 minutes. The mixture was stirred at room temperature (20-24° C.) for 20.5 hours. After 20.5 hours, the reaction mixture was gradually added (during 1 minute) while stirring, to 0.75 liter of cold water at 15° C., leading to precipitation of a white solid lump. The product was separated by filtration through Buchner and washed with 1 liter of water. The product was dried under reduced pressure in a vacuum oven at 50° C. to yield 4 g of a white solid. The dry solid was grinded (intermediate I).

Step 2:

A 19% solution of HBr in AcOH was prepared by adding 0.11 liter of a solution of 33% hydrogen bromide in acetic acid to 0.11 liter of AcOH. The resulting solution was added to the grinded intermediate from step I while stirring at ˜200 rpm. After 2 hours the stirring was increased to 400 rpm and continued for 4 hours at room temperature. The clear solution was gradually added (during 1 minute), to 0.75 liter of water at 10° C., while stirring leading to the formation of white precipitate. The product was separated by filtration using Buchner and washed with 0.7 liter of water. The product was dried under reduced pressure at 25° C. in a vacuum oven (intermediate II).

Step 3:

Intermediate II was added to 0.22 liter of water in 0.5 round bottomed flask followed by addition of 25 ml of piperidine. The mixture was stirred at room temperature for 22 hours. The mixture became clear after less than 2 hours of stirring. The mixture was then filtered through a filter paper. The paper was washed with 50 ml of water to produce clear and colorless solution. Water was added up to 0.35 liter through the filter resulting in ˜6.6% piperidine solution.

Step 4:

The crude product in solution was purified by Ultrafiltration/diafiltration using two 0.093 m² suspended screen PES Pall cassettes, cutoff 5K. The reaction solution was concentrated to 0.2 liter and diafiltration at transmembrane pressure (TMP) 2 bar, feed flow 100 liter/hour, was performed against 1.2 liters of water followed by 0.6 liters of 0.3% AcOH solution and finally with 0.6 liters of water. The purified solution was freeze dried resulting in 1.8 g of solid with an average molecular weight of 34,181 Dal.

The synthesized copolymers YB-105, YB-106, YB-107 and YB-108 were analysed by SEC-HPLC on a cross-linked, agarose-based medium column, Superose 12 10/300GL (GE Healthcare). The analysis was performed at a flow rate of about 0.4 ml/min at isocratic mode, the mobile phase comprised 80% 50 mM potassium phosphate and 150 mM potassium chloride buffer and 20% acetonitrile, run time of about 80 min.

Table 1 hereinbelow presents the size exclusion analyses of the synthesized copolymers YB-105, YB-106, YB-107 and YB-108.

TABLE 1
SEC-HPLC Analysis of the synthesized copolymers

| Batch No. | RT, min | W_{1/2}, min | T_{USP} |
|---|---|---|---|
| YB-123 | 34.73 | 9.57 | 0.97 |
| (reference sample) | | | |
| YB-105 | 30.06 | 12.09 | 1.01 |
| YB-106 | 24.90 | 11.19 | 1.82 |
| YB-107 | 44.14 | 5.12 | 1.04 |
| YB-108 | 31.12 | 11.48 | 1.12 |

### Example 2

### Standard Potency Assay Protocol

Short Description of the Method

Generally, following immunization, T cells of the immune system recognize immunogenic peptides presented by class I or class II major histocompatibility complex (MHC) molecules, expressed on antigen presenting cells (APC). The specificity of antigen recognition by T cells is defined by the affinity of the T cell receptor to the MHC-peptide complex as well as the primary sequence of the antigenic peptide. Following antigen recognition, clonal expansion of the specific T cells occurs. A second stimulus, causes differentiation and secretion of a variety of cytokines, which are typical of the expanded T-cell population. Proliferating helper T cells, which develop into effector T cells, differentiate into two major subtypes of cells known as Tₕ1 and Tₕ2 cells, each subtype interacts with a different cell partner and secretes a typical set of cytokines.

The present inventors developed an ex-vivo biological potency assay to determine the relative potency of GA based on effecting the first stimulus by immunizing mice in-vivo with Poly-YAK (also referred to as activation), and the second stimulus by in-vitro stimulation (also referred to as stimulation) with GA. The assay is composed of 6 major steps depicted in FIG. 1.

Briefly, mice are immunized with a pre-determined amount of Poly-YAK (YB106), mixed 1:1 with CFA (step 1). Eight to ten days later, the mice are sacrificed and draining lymph nodes are harvested. Lymph node cells (LNCs) are extracted from the lymph nodes and counted (step 2). The cells are then incubated with different amounts of GA Reference Standard (RS), positive control (Concavalin A, nonspecific activator of T-cells), negative controls (MBP peptides), or samples of a tested batch of GA (step 3). Following ˜23 hours (between 19 to 27 hours) of incubation the conditioned media is collected and kept frozen for further analysis. The Level of IL-2 secretion from the cells into the culture media is determined by a commercial IL-2 ELISA (step 4).

In order to determine the relative potency of a tested batch, IL-2 secretion by the LNCs incubated with the tested batch samples is compared to the IL-2 secretion by the LNCs incubated with RS samples (step 5). Parallelism of the RS and tested batch curves and potency of the tested batch are determined accordingly (step 6).

Materials and Methods

Synthesis of Poly-YAK—as described in Example 1 hereinabove.

Animals—Female (SJLXBALB/C) F1 mice, 8 to 12 weeks old (Harlan Laboratories Ltd. Israel) were used. Animal housing and care conditions were maintained in conventional manner under controlled environmental conditions and veterinary supervision.

Immunization—Poly-YAK (YB106) was dissolved to final concentration of 20 mg/ml in 40 mg/ml Mannitol (G. T. Baker, Cat. #2553-01) and adjusted to pH 6-7 with acetic acid. Poly-YAK (YB106) at 5 mg/ml was prepared by dilution 1:2 in sterile PBS (Sigma, Cat. # P3813) and mixed 1:2 with CFA (Sigma, Cat. # F5881) (total dilution of 1:4). An emulsion was created by transferring the mixture between two glass syringes through a luer bridge. 50 μl of the emulsion was injected into the left footpad of each mouse (final 250 μg/mouse).

Lymph node cells (LNCs) extraction—The primary culture of LNCs was prepared 8 to 10 days following immunization. Mice were sacrificed and the popliteal lymph nodes (LN) were harvested in cold RPMI medium. The LNs were transferred into a sterile 3 cm petri dish containing about 3 ml sterile RPMI medium and forced through a 100μ mesh cell strainer (BD, Falcon Cat. No. 352360) by a sterile 2.5 ml syringe plunger. The cells were then collected in 10-20 ml RPMI or AIM-V complete medium and counted by hemocytometer using Erythrosine B (Sigma, cat no. E9259). The cells were centrifuged at 220 g for 10 minutes at room temperature and re-suspended to the required density in AIM-V complete medium.

In vitro stimulation—The in vitro stimulation was performed in 96 wells plates. The RS (COPAXONE®-Teva, batches P53668 and P53824) and tested samples were diluted at the required concentrations (final range on plate of 2-80 μg/ml) in AIM-V or RPMI complete medium. An example of RS and tested samples preparation is presented in table 4 below. Serial dilutions were performed in 3 independent triplicates; the 1^{st} dilution was conducted in LoBind tubes (Eppendorf) while all other dilutions were conducted in polypropylene “U” 96 wells plates. 2.5 μg/ml ConA (Sigma, Cat. No. C-5275) was used as Positive control (PC), and 20 μg/ml MBP peptide (BACHEM, Cat. No. H-1964) was used as negative control (NC), were prepared in AIM-V or RPMI complete medium. Triplicates of 100 μl positive and negative controls and diluted RS and tested samples were dispensed in 96 wells tissue culture plate. An example of the 96 wells plate set up is presented in table 5 below. 100 μl (5*10⁶/ml) LNCs were then added to each well and the plates were incubated for ˜23 hours in 5% CO₂ humidified incubator at 37° C.

TABLE 2
Example of RS and tested samples preparation.

| | | | | | | Final on |
|---|---|---|---|---|---|---|
| | | Dilution | Take | Medium | Final | plate |
| Dilution # | Stock | factor | (μl) | (μl) | (μg/ml) | (μg/ml) |
| 1 | 20 mg/ml | 125 | 10 | 1240 | 160 | 80 |
| 2 | 80 μg/ml | 0.6 | 210 | 140 | 96 | 48 |
| 3 | 48 μg/ml | 0.6 | 210 | 140 | 57.6 | 28.8 |
| 4 | 28.8 μg/ml | 0.6 | 210 | 140 | 34.6 | 17.3 |
| 5 | 17.3 μg/ml | 0.6 | 210 | 140 | 20.8 | 10.4 |
| 6 | 10.4 μg/ml | 0.6 | 210 | 140 | 12.4 | 6.2 |
| 7 | 6.2 μg/ml | 0.6 | 210 | 140 | 7.4 | 3.7 |
| 8 | 3.7 μg/ml | 0.6 | 210 | 140 | 4.4 | 2.2 |

TABLE 3
Example of a 96 wells plate set up.

| | | RS | | | Sample #1 | | | Sample #2 | | | | |
|---|---|---|---|---|---|---|---|---|---|---|---|---|
| | 1 | 2 | 3 | 4 | 5 | 6 | 7 | 8 | 9 | 10 | 11 | 12 |
| A | | 80.0 | 80.0 | 80.0 | 80.0 | 80.0 | 80.0 | 80.0 | 80.0 | 80.0 | 0 | |
| B | | 48.0 | 48.0 | 48.0 | 48.0 | 48.0 | 48.0 | 48.0 | 48.0 | 48.0 | 0 | |
| C | | 28.8 | 28.8 | 28.8 | 28.8 | 28.8 | 28.8 | 28.8 | 28.8 | 28.8 | NC | |
| D | | 17.3 | 17.3 | 17.3 | 17.3 | 17.3 | 17.3 | 17.3 | 17.3 | 17.3 | NC | |
| E | | 10.4 | 10.4 | 10.4 | 10.4 | 10.4 | 10.4 | 10.4 | 10.4 | 10.4 | NC | |
| F | | 6.2 | 6.2 | 6.2 | 6.2 | 6.2 | 6.2 | 6.2 | 6.2 | 6.2 | PC | |
| G | | 3.7 | 3.7 | 3.7 | 3.7 | 3.7 | 3.7 | 3.7 | 3.7 | 3.7 | PC | |
| H | | 2.2 | 2.2 | 2.2 | 2.2 | 2.2 | 2.2 | 2.2 | 2.2 | 2.2 | PC | |

Collection of conditioned media—At the end of the experiment, the plates were centrifuged at 200-250 g for 10 minutes at 4° C. and the conditioned media was collected. From each well, two duplicates of 75 μl each were transferred to two polypropylene U bottom 96 wells plates for storage at −80° C.

Quantitative determination of IL-2 level—IL-2 ELISA was preformed according to the manufacturer's instructions (BD OptEIA™, mouse IL2 ELISA, Cat. No., Cat. No. 55148). All reagents were used at room temperature. Standard recombinant IL-2 was suspended in 1 ml DDW, aliquoted to 20 μl and stored at −80° C., according to manufacturer's instructions. For each ELISA plate a GA Reference standard (RS) curve was generated representing the levels of IL-2 secretion in response to elevating concentrations of RS. Each test sample curve was then compared to the RS curve from the same plate. A representative RS curve in which IL-2 responses are presented by a log/log graph, wherein the x axis stands for the stimulator (RS) concentration (μg/ml) and the y axis for the IL-2 levels (μg/ml) is demonstrated in FIG. 2.

Calculations

Calculations were performed using the following softwares: SoftMaxPro5, Excel, Prizm5 and PLA (parallel line analysis).
IL-2 level for each sample was determined using SoftMaxPro5.
For each data point the logarithm of both stimulator (e.g.; RS, COPAXONE® tested batch, GA R&D tested batch, Specificity sample) concentration and IL-2 concentration was calculated using Prizm5.
A plot of Log [IL-2 average] against Log [stimulator] was generated.
The slope of RS and tested samples curves were determined and parallel slopes were checked at 95% confidence.
The combined slope for all data points (RS and tested samples) was calculated using Excel according to:

β*=Σ(Y−Yave)*(X−Xave)/Σ(X−Xave)
β*—RS and tested sample combined slope
Y—Log [IL-2], X— Log [stimulator] is for each data point
Yave, Xave—Y average, X average for all data points
The relative potency was determined using PLA software according to:

% Potency=100*10̂{((YaveSa−YaveRS)/β*−(SaveSa−XaveRS)}

YaveSa is the average Log [IL-2] of all tested sample points
YaveRS is the average Log [IL-2] of all RS points
XaveSa is the average Log [stimulator] of all tested sample points
XaveRS is the average Log [stimulator] of all RS points

Ex-Vivo Potency Assay Acceptance Criteria

### GA Reference Standard (RS) Acceptance Criteria:

Curve should include at least 5 concentrations
Slope≥0.8
Curve fit R² ≥0.95

### Tested Sample Acceptance Criteria Include the Above and in Addition:

Sample curve should be parallel at 95% confidence to the RS curve
The calculated potency of the tested sample should be in the range of 80-125% of the RS.

### Example 3

### Immunization Using 3 Amino Acids Copolymers Followed by In-Vitro Stimulation with GA: Yb106 is Superior

Materials and Methods:

Syntheses of Poly-EAK, Poly-YAK, Poly-YEA, and Poly-YEK—as described in Example 1 hereinabove.

Immunization—Immunization with Poly-YAK Poly-EAK, Poly-YEA or Poly-YEK was performed as described in Example 2 hereinabove.

Lymph node cells (LNCs) extraction—as described in Example 2 hereinabove.

In-vitro stimulation—as described in Example 2 hereinabove.

Collection of conditioned media—as described in Example 2 hereinabove.

Quantitative determination of IL-2 level—as described in Example 2 hereinabove.

Results

In order to test the ability of the synthesized copolymers comprising 3 amino acids (Poly-EAK, Poly-YAK, Poly-YEA, and Poly-YEK) to activate in-vivo lymph node cells (LNCs) that respond to GA in-vitro stimulation, each of the synthesized copolymers was used for immunization of mice at 250 μg/mouse. LNCs were extracted from sacrificed mice nine days after immunization. The extracted LNCs were then stimulated in-vitro with GA Reference Standard (RS, COPAXONE®-Teva, batch P53668) and the response was evaluated by IL-2 secretion to the culture media.

As demonstrated in FIG. 3, in-vivo immunization using Poly-YAK (YB106) resulted in activation of LNCs that responded to in-vitro stimulation with RS by secretion of IL-2 in a dose-dependent manner, with a high slope (>0.8) and a good linear regression fit (R²=0.99).

FIG. 3 also demonstrates that in-vivo immunization using Poly-EAK (YB105) resulted in activation of LNCs that responded to in-vitro stimulation with RS by secretion of IL-2 in a dose-dependent manner, however with a lower slope and lower linear regression fit than that observed with Poly-YAK (YB106).

Furthermore, FIG. 3 demonstrates that in-vivo immunization using Poly-YEA (YB107) failed to activate LNCs that can respond to in-vitro simulation with RS, as evident by low IL-2 secretion in all RS concentrations tested.

On the other hand, in-vivo immunization using Poly-YEK (YB108) resulted in activation of LNCs that responded to in-vitro stimulation with RS by secretion of high levels of IL-2 in all RS concentrations tested. However, no dose response relationship was observed as evidenced by a very low slope (0.51±0.01).

Summary

Taken together, the curve slope, linearity and intercept of the IL-2 secretion response indicated that Poly-YAK (YB106) is the preferred copolymer for in-vivo immunization followed by in-vitro stimulation with RS.

### Example 4

### Potency Assay Characterization

Materials and Methods

All materials and methods as described in Examples 1 and 2 hereinabove.

Results

Specificity

Specificity of the ex-vivo potency assay was tested by performing the in-vitro stimulation of LNCs extracted from mice immunized with Poly-YAK (YB106) with the different specificity samples YB105, YB106, YB107, YB108, YB109 and YB110 and with GA reference standard (RS, COPAXONE®-Teva, batches P53668 and P53824). As clearly demonstrated in FIGS. 4A-F, the potency assay was able to distinguish between the different samples, each having its own pattern of IL-2 secretion.

Specifically, YB105 (Poly-EAK) had a lower IL-2 response which was parallel to the RS response only in the top curve concentrations (FIG. 4A). YB107 (Poly-YEA) demonstrated a lower IL-2 response non parallel to RS curve. YB106 (Poly-YAK) had an IL-2 response only at low concentrations followed by a sharp decrease in IL-2 secretion. IL-2 response to YB108 (Poly-YEK) treatment was constantly lower than the RS response, not parallel, and decreased at high concentrations. Stimulation with YB109, (a short GA form with molecular weight ˜5Kdal) resulted in a linear IL-2 secretion curve, which was parallel to RS but slightly lower (calculated potency 69%). Treatment with YB110, a long GA form with molecular weight ˜35Kdal, led to a high IL-2 secretion response, which decreased at high concentrations and wasn't parallel to RS. Stimulation with irrelevant MBP peptide (87-99) used as a negative control did not induce IL-2 secretion response.

Robustness

In-Vivo Activation Period

To test the robustness of the in-vivo activation period, mice were sacrificed at days 8, 9 and 10 after immunization. As demonstrated in FIG. 5A, IL-2 secretion was identical in all 3 days of sacrifice tested, indicating that the assay is robust for 9±1 days of in-vivo activation. In addition, the present inventors have tested the effect of additional in-vivo activation periods. As demonstrated in FIG. 5B, In-vivo activation of 3, 9, or 14 days resulted in extraction of LNCs that responded in a similar manner to in-vitro stimulation with RS by secretion of IL-2 in a dose-dependent manner, with the same slope (=1) and a good linear regression fit (R²≥0.92), indicating that the in-vivo activation step can be effected for 3 to 14 days.

Immunization Dose

To test the robustness of the Poly-YAK (YB106) immunization dose, mice were immunized with 100 μg/mouse or 250 μg/mouse of the synthesized Poly-YAK (YB106). As demonstrated in FIG. 6A, in-vivo immunization using 100 μg/mouse or 250 μg/mouse Poly-YAK (YB106) resulted in activation of LNCs that responded in a similar manner to in-vitro stimulation with RS by secretion of IL-2 in a dose-dependent manner, with a slope (>0.8) and a good linear regression fit (R²=0.99), indicating that the assay is robust for 100 μg/mouse and 250 μg/mouse Poly-YAK immunization doses.

In-Vitro Stimulation Period

To test the robustness of the In-vitro stimulation period, the in-vitro stimulation with RS and mock RS accuracy samples (COPAXONE®-Teva, batch P53668 or P53824) was conducted for 23±2 hours. As demonstrated in Table 4 hereinbelow, similar IL2 secretion curves were accepted upon stimulation with the RS and the accuracy samples in all in-vitro stimulation periods tested, indicating that the assay is robust for 23±2 hours of in-vitro stimulation. In addition, the present inventors have tested the effect of additional in-vitro stimulation periods. In-vitro stimulation of 17-27 hours resulted in secretion of IL-2 in a dose-dependent manner, with a nice slope and a good linear regression fit, indicating that the in-vitro stimulation step can be effected for 17-27 hours.

TABLE 4
Robustness of the in-vitro stimulation period.

| stimulation | | | | Calculated |
|---|---|---|---|---|
| time (hours) | Sample | Slope | Intercept (X = 0) | Potency (%) |
| 21 | RS | 0.95 | 0.8 | — |
| | RS 75% | 0.95 | 0.7 | 72.3 |
| | RS 125% | 0.93 | 0.9 | 115.2 |
| 23 | RS | 0.91 | 0.8 | — |
| | RS 75% | 0.91 | 0.7 | 72.8 |
| | RS 125% | 0.88 | 1.0 | 120.7 |
| 25 | RS | 0.94 | 0.8 | — |
| | RS 75% | 0.95 | 0.7 | 72.4 |
| | RS 125% | 0.95 | 0.9 | 128.5 |

Accuracy

To evaluate accuracy of the developed potency assay, mock concentrations of RS (COPAXONE®-Teva, batch P53668 or P53824) in a range of 50%-200% were used as in-vitro stimulators. The results indicated that the assay is accurate at the range of 50%-200% GA with less than 5% inaccuracy of the calculated relative potency for all the mock RS accuracy samples. As demonstrated in FIG. 6B, linearity of the assay at the range of 50-200% was demonstrated with R²=0.99.

Potency Evaluation of Different GA Batches

Test of Multiple COPAXONE® Batches

Several COPAXONE® batches were tested for their biological potency using the ex-vivo assay in comparison to RS (COPAXONE®-Teva, batch P53668). All tested COPAXONE® batches were within their labeled shelf-life. As demonstrated in FIGS. 7A-F and in Table 5 hereinbelow, all COPAXONE® batches had an IL-2 response parallel to the IL-2 response of the RS with potency ranging between 88% and 109% in comparison to the RS. % CV of repeated determinations of the same COPAXONE® batch were ≤14%.

TABLE 5
Summary the potency results of COPAXONE ® batches.

| Batch | potency % | Average potency % |
|---|---|---|
| P53714 | 96 | 98 |
| | 99 | |
| P53791 | 97 | 97 |
| P53459 | 102 | 102 |
| P53551 | 107 | 107 |
| P53824 | 95 | 102 |
| | 109 | |
| P53889 | 93 | 93 |
| P53804 | 102 | 102 |
| P53767 | 92 | 92 |
| P53893 | 90 | 99 |
| | 88 | |

Test of Multiple GA Non-Commercial Batches

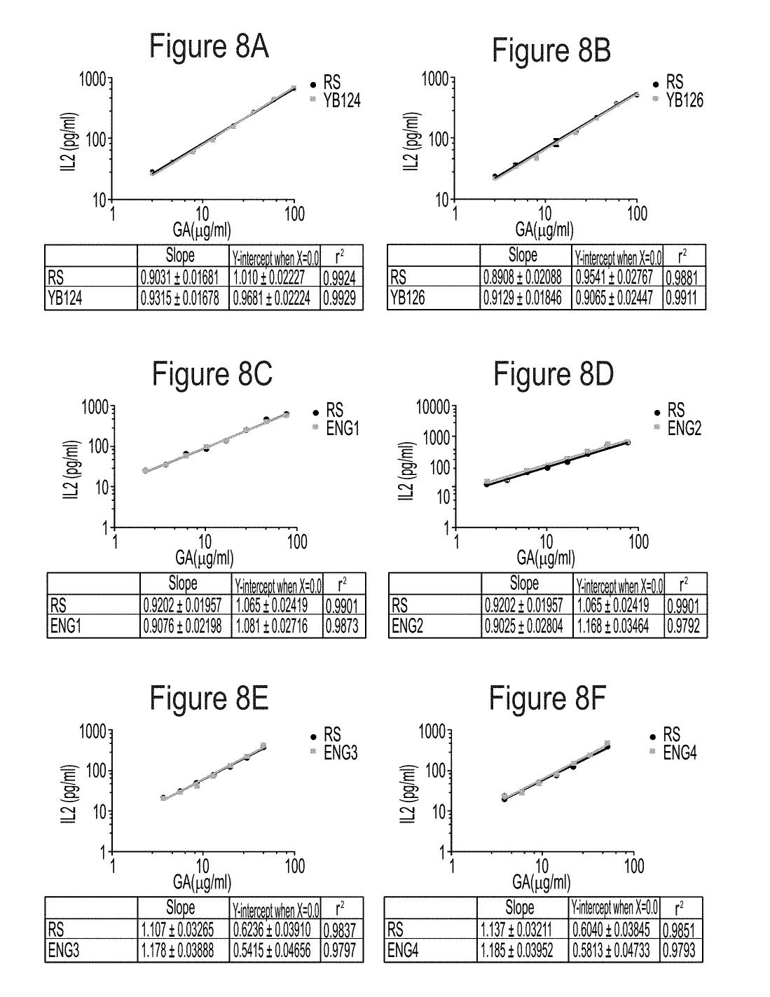

Several GA non-commercial_batches were tested for their biological potency using the ex-vivo assay in comparison to RS (COPAXONE®-Teva, batches P53668 or P53824); these include batches from R&D scale, pilot scale and engineering batches manufactured at production scale. As demonstrated in FIGS. 8A-F and in Table 6 hereinbelow, all tested GA batches had an IL-2 response parallel to the IL-2 response of the RS with potency ranging between 89% and 110% in comparison to the RS.

TABLE 6
Summary the potency results of GA non-commercial batches.

| Sample | Average Potency % |
|---|---|
| YB124P | 96 |
| YB124D | 94 |
| YB126 | 89 |
| YB123 | 95 |
| ENG1 | 92 |
| ENG2 | 110 |
| ENG3 | 99 |
| ENG4 | 109 |

Summary

Taken together, the ex-vivo potency assay developed by the present inventors demonstrated a strong dose-dependent in-vitro IL-2 secretion in response to GA. The results indicate that the assay is highly specific to stimulation with GA, does not respond to non-related antigens and sensitive to changes in the amino acids composition as well as to changes in the average molecular weight of GA. The robustness results demonstrated that the in-vitro activation period can vary between 8 to 10 days and the immunization dose can vary from 100 μg/mouse to 250 μg/mouse without affecting the assay results. Most importantly, the assay was able to show acceptable levels of relative potency of several COPAXONE® batches as well as GA non-commercial batches, passing the acceptance criteria of the assay.

### Example 5

### Profile of Cytokine Secretion

Materials and Methods:

Syntheses of Poly-YAK—as described in Example 1 hereinabove.

Immunization—as described in Example 2 hereinabove.

Lymph node cells (LNCs) extraction—as described in Example 2 hereinabove.

In-vitro stimulation—as described in Example 2 hereinabove in 24 wells plates with 50 μg/ml RS (COPAXONE®-Teva, batch P53824), 50 μg/ml GA non-commercial batch ENG2 and non-stimulated cells (NC).

Collection of conditioned media—as described in Example 2 hereinabove with modifications to 24 wells plates.

Cytokine secretion profile—Cytokine secretion profile was determined using an antibody array specific for mouse cytokines according to the manufacturer's instructions (Mouse Cytokine Array Panel A, array kit—R&D Systems). Briefly, conditioned medium from GA (50 μg/ml) stimulated or from not stimulated (Negative control, NC) cells was collected, clarified by centrifugation and incubated for ˜20 hours with Mouse Cytokine Array Panel A membrane. Cytokines attached to the membranes were detected according to manufacturer instructions. The membranes were developed using chemiluminescence and the developed signal was captured using GS-800 calibrated densitometer (BIO-RAD). Intensity of specific array dotes was relatively quantitated using Quantity One software. For numerical calculations each pair of duplicate dots was averaged.

Results

In order to characterize the lymphocyte response of Poly-YAK (YB106) activated cells, mice were immunized with YB106 and lymph nodes were harvested nine days later. Extracted LNCs were then stimulated in-vitro with RS COPAXONE®-Teva, batch P53824) or GA non-commercial batch ENG2 and the cytokine secretion profile as compared to non-stimulated cells (NC) was determined using an antibody array specific for mouse cytokines.

The densitometry results, as summarized in Table 7 herein below, demonstrate that the major cytokines secreted from LNCs in response to GA stimulation were GM-CSF, 1-309, IFNγ, IL-2, IL-3, IP-10, and MIP-1α.

TABLE 7
Summary of the densitometry numerical
results of the cytokine array

| Cytokine | control-NC | P53824 | ENG2 |
|---|---|---|---|
| GM-CSF | 1.1 | 6.9 | 6.9 |
| I-309 | 1.0 | 5.8 | 6.0 |
| sICAM-1 | 1.5 | 2.3 | 3.1 |
| IFN-γ | 1.4 | 3.3 | 4.0 |
| IL-1ra | 1.1 | 1.1 | 1.3 |
| IL-2 | 1.0 | 5.2 | 5.9 |
| IL-3 | 1.0 | 6.2 | 6.5 |
| IL-10 | 1.0 | 1.8 | 1.7 |
| IL-13 | 1.0 | 1.1 | 1.2 |
| IL-16 | 1.0 | 1.0 | 0.9 |
| IL-17 | 1.0 | 2.7 | 3.5 |
| IP-10 | 1.0 | 7.8 | 8.7 |
| MIG | 1.0 | 1.1 | 1.3 |
| MIP-1α | 1.1 | 7.2 | 7.7 |
| MIP-1β | 1.0 | 1.9 | 2.3 |
| RANTES | 1.0 | 1.2 | 1.5 |

Summary

Taken together, the cytokine secretion profile results indicate that the LNCs, generated according to the present method, secrete several cytokines that can be quantified as a measure for the potency of GA.

### Example 6

### Immunization Using GA Followed by In-Vitro Stimulation with 3 Amino Acids Copolymers

Materials and Methods

Animals—As described in Example 2 hereinabove.

Immunization—GA reference standard (RS, COPAXONE®-Teva, batch P53435) at 4 mg/ml was prepared by dilution 1:5 in sterile PBS and mixed 1:2 with Complete Freund's Adjuvant (CFA, Sigma, Cat. # F5881). An emulsion was created by transferring the mixture between two glass syringes through a loar bridge. 50 μl of the emulsion was injected into the left footpad of each mouse (final 100 μg RS/mouse).

Lymph node cells (LNCs) extraction—The primary culture of LNCs was prepared 9 to 11 days following immunization as described in Example 2 hereinabove.

In vitro stimulation—The in vitro stimulation was performed as described in Example 2 hereinabove, using RS (COPAXONE®-Teva, batch P53435) and tested samples at final concentration range on plate of 2.5-50 μg/ml.

Collection of conditioned media—As described in Example 2 hereinabove.

Quantitative determination of IL-2 level—As described in Example 2 hereinabove.

Calculations—Calculations were performed using the following softwares: SoftMaxPro5, Excel, Prizm5 and PLA.
IL-2 level for each sample was determined using SoftMaxPro5.
For each data point the logarithm of RS concentration and IL-2 concentration was calculated using Prizm5.
A plot of Log [IL-2 average] against Log [RS] was generated.
The slope of RS and tested samples curves were determined and parallel slopes were checked at 95% confidence.

Results

In order to test the ability of the synthesized copolymers comprising 3 amino acids (Poly-EAK, Poly-YAK, Poly-YEA, and Poly-YEK) to stimulate in-vitro GA specific lymph node cells (LNCs) generated by in-vivo immunization with GA, each of the synthesized copolymers was used for in-vitro stimulation at increasing concentrations. Briefly, mice were immunized with GA reference standard (RS) and LNCs were extracted from sacrificed mice 9-11 days after immunization. The extracted LNCs were then stimulated in-vitro with RS (COPAXONE®-Teva, batch P53435), Poly-EAK (YB105), Poly-YAK (YB106), Poly-YEA (YB107), or Poly-YEK (YB108) and the response was evaluated by IL-2 secretion to the culture media.

As demonstrated in FIGS. 9A-B, in-vitro stimulation using Poly-YAK (YB106) demonstrated no dose-response relationship between the stimulator and IL-2 secretion. In-vitro stimulation using Poly-YEK (YB108) demonstrated a non-linear dose response relationship between the stimulator and IL-2 secretion contrary to RS stimulation. In-vitro stimulation using Poly-EAK (YB105) or Poly-YEA (YB107) demonstrated a linear effect on IL-2 secretion on IL-2 secretion that was much lower and not parallel to the RS stimulation curve.

Summary

All four (Poly-EAK, Poly-YAK, Poly-YEA and Poly-YEK) copolymers did not induce in-vitro stimulation of LNCs from GA immunized mice that was comparable to the in-vitro stimulation by the GA reference standard (RS). The results obtained are summarized in Table 7 hereinbelow.

TABLE 8
Summary of the ex-vivo potency tests comprising immunization with
GA and in-vitro stimulation with 3 amino acids copolymers

| Sample | | |
|---|---|---|
| name | description | Potency test result as evaluated by IL-2 secretion |
| YB105 | Poly-EAK | Lower response not parallel to RS |
| YB106 | Poly-YAK | No linear fit, similar to RS response at low con- |
| | | centration, fall down at 10 μg/ml |
| YB107 | Poly-YEA | Lower response not parallel to RS |
| YB108 | Poly-YEK | No linear fit, low response at low concentration, |
| | | up to 30 μg/ml |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of determining relative potency of a batch of glatiramer acetate (GA), the method comprising:
(a) immunizing a mammal with an immunization effective amount of a Poly-YAK copolymer (pYAK) so as to obtain an immunized mammal;
(b) preparing a primary culture of T-cells from said immunized mammal;
(c) individually stimulating samples comprising substantially identical number of cells of said primary culture of T-cells with a predetermined amount of:
(i) a reference standard (RS) batch of GA; or
(ii) the batch of GA,
wherein said predetermined amount of (i) is substantially identical to said predetermined amount of (ii);
(d) determining a stimulation parameter of said cells in each said samples following a predetermined incubation time; and
(e) comparing said stimulation parameter in said cells of step (c)(i) with said stimulation parameter of said cells of step (c)(ii), so as to determine the relative potency of the batch of GA.

2. The method of claim 1, further comprising generating stimulation curves of said samples incubated with predetermined amount of said RS batch of GA and of said samples incubated with predetermined amount of said batch of GA, so as to determine parallelism.

3. The method of claim 1, wherein said mammal is a rodent.

4-6. (canceled)

7. The method of claim 1, wherein said immunization effective amount of pYAK comprises 100 to 250 μg per mammal per boost.

8. The method of claim 1, wherein said immunizing comprises administering an effective amount of an adjuvant simultaneously with said Poly-YAK copolymer (pYAK).

9. (canceled)

10. The method of claim 1, wherein said preparing a primary culture of T-cells is effected 3 to 14 days following immunizing said mammal.

11. The method of claim 1, wherein said T-cells are lymph node cells.

12. The method of claim 1, wherein said T-cells are spleen cells.

13. The method of claim 1, wherein said samples of each of said (c)(i) and (c)(ii) comprise at least 2 repeats.

14. The method of claim 1, wherein said samples of each of said (c)(i) and (c)(ii) comprise at least 3 repeats.

15. The method of claim 1, wherein said predetermined amount of said RS batch of GA and the batch of GA comprise 2 μg/ml to 80 μg/ml.

16. The method of claim 1, wherein said stimulation parameter is cytokine secretion.

17. The method of claim 16, wherein said cytokine is an interleukin.

18. (canceled)

19. The method of claim 1, wherein said predetermined incubation time comprises 17 to 27 hours.

20. A method of preparing a batch of glatiramer acetate as acceptable for pharmaceutical use, the method comprising:
(a) preparing a batch of GA;
(b) measuring the relative potency of the batch according to the method of claim 1; and
(c) qualifying the batch as acceptable for pharmaceutical use if the relative potency so measured in step (b) is between 80% and 125% of the RS batch of GA.

21. The method of claim 20, further comprising generating stimulation curves of said samples incubated with predetermined amount of said RS batch of GA and of said samples incubated with predetermined amount of said batch of GA, and qualifying the batch as acceptable for pharmaceutical use if said stimulation curves are parallel.

22. A method of preparing a pharmaceutical composition comprising GA, the method comprising:
(a) preparing a batch of GA;
(b) measuring the relative potency of the batch according to the method of claim 1; and
(c) preparing the pharmaceutical composition with the batch of GA if the relative potency so measured in step (b) is between 80% and 125% of the RS batch of GA.

23. The method of claim 22, further comprising generating stimulation curves of said samples incubated with predetermined amount of said RS batch of GA and of said samples incubated with predetermined amount of said batch of GA, and preparing the pharmaceutical if said stimulation curves are parallel.

24. A method of treating multiple sclerosis (MS) in a subject in need thereof, the method comprising injecting into the subject a pharmaceutical composition which comprises GA generated according to the method of claim 22, thereby treating MS in a subject.
